(19) United States
(12) Patent Application Publication
(10) Pub. No.: US 2025/0285269 A1
(43) Pub. Date: Sep. 11, 2025

(54) SYSTEMS AND METHODS FOR HEMOGLOBIN LEVEL DETERMINATION

(71) Applicant: MONERE CORPORATION, Lehi, UT (US)

(72) Inventors: Mou NANDI, Lehi, UT (US); Krishanu BANERJEE, Lehi, UT (US); Jhuma NANDI, Belle Mead, NJ (US)

(21) Appl. No.: 19/069,080

(22) Filed: Mar. 3, 2025

Related U.S. Application Data
(60) Provisional application No. 63/563,023, filed on Mar. 8, 2024.

(51) Int. Cl. G06T 7/00; A61B 3/14; A61B 5/00; A61B 5/145; A61B 5/1455; G06T 5/60; G06T 5/70; G06T 7/11; G06T 7/90; G06V 10/764; G06V 10/766; G06V 10/82; G06V 10/94; G06V 20/70; G06V 40/18

(52) U.S. Cl. CPC G06T 7/0012; A61B 3/14; A61B 5/14546; A61B 5/1455; A61B 5/7203; A61B 5/7267; G06T 5/60; G06T 5/70; G06T 7/11; G06T 7/90; G06V 10/764; G06V 10/766; G06V 10/82; G06V 20/70; G06V 40/193; G06V 40/197; A61B 2576/02; G06T 2207/20076; G06T 2207/20081; G06T 2207/30041; G06T 2207/30201; G06V 10/95

(57) ABSTRACT

Disclosed herein are systems, methods, computer-readable media, and techniques for determining health indicators from images taken using a non-augmented mobile device. Methods are presented for processing images comprising the lower eyelid portion of an eye and determining levels of said health indicators using one or more machine learning algorithms. One such health indicator is hemoglobin, the level of which can be used to determine at least indications of general health or presence of an anemia condition in a subject.

### CROSS-REFERENCE TO RELATED APPLICATIONS

This application claims the benefit of U.S. Application No. 63/563,023, filed Mar. 8, 2024, each of which is incorporated herein by reference in its entirety.

Non-invasive, accessible diagnostic tools are valuable to providing health monitoring and preventing or treating illness early. These may be especially valuable for regions with low access to medical resources. Hemoglobin levels may be a useful indicator of health that is expected to have relatively stable values over time. Deviations from a baseline value can indicate the presence of ailments such as anemia. Existing tools for measuring hemoglobin levels require one or both of invasive techniques (e.g., blood draw) or specialized equipment (e.g., optical reflectance photometer).

### SUMMARY

In one aspect disclosed herein is a computer-implemented method for analyzing an image of an eye, comprising: obtaining the image of the eye; and providing a machine learning pipeline configured to perform operations comprising: identifying a lower eyelid portion in the image of the eye, and determining a hemoglobin level from an input comprising the lower eyelid portion. In some cases, the image of the eye is obtained using a non-augmented mobile device camera. In some cases, the machine learning pipeline is further configured to perform adjusting a white balance of the image of the eye using a sclera portion of the eye. In some cases, the machine learning pipeline is further configured to perform denoising the image of the eye. In some cases, the machine learning pipeline is further configured to perform excluding the image of the eye in response to the image of the eye comprising a red value or a yellow value satisfying a threshold. In further embodiments, the red value satisfying the threshold corresponds to an indication of conjunctivitis. In further embodiments, the yellow value satisfying the threshold corresponds to an indication of jaundice. In some cases, the identifying the lower eyelid portion operation comprises a classification operation and a segmentation operation. In further embodiments, the classification operation comprises predicting a presence of the lower eyelid portion in the image of the eye. In further embodiments, the segmentation operation comprises identifying a plurality of pixels corresponding to the lower eyelid portion of the image of the eye. In some cases, the hemoglobin level is within about 0.93 grams per deciliter to about 1.1 grams per deciliter of a true value. In some cases, the hemoglobin level is within about 1.56 grams per deciliter of a true hemoglobin level. In some cases, the hemoglobin level is within about 0.93 grams per deciliter of a true hemoglobin level. In some cases, the input further comprises demographic data of a subject. In further embodiments, the demographic data of the subject comprises one or more of age, sex, or pregnancy status. In some cases, the input further comprises a medical history of a subject. In further embodiments, the medical history comprises one or more of smoking, cancer, chemotherapy, chronic kidney disease, body temperature, fatigue, blood pressure, or diabetes. In some cases, the medical history of the subject comprises multi-modal data. In yet further embodiments, the multi-modal data comprises one or both of an image data or textual data. In some cases, the method further comprises providing the hemoglobin level to a user device. In further embodiments, the user device is a smartphone. In yet further embodiments, the user device is a computing device of a healthcare provider. In some cases, the method further comprises obtaining the image of the eye using a user device. In yet further embodiments, the user device is a non-augmented mobile device. In some cases, the hemoglobin level is used to generate a care plan. In further embodiments, the care plan comprises one or more suggestion to improve the hemoglobin level. In some cases, the hemoglobin level is used to diagnose a subject with anemia. In some cases, the machine learning pipeline is further configured to determine a bilirubin level from the input comprising the lower eyelid portion.

In another aspect disclosed herein is a computer-implemented method for analyzing an image of an eye, comprising: obtaining, by one or more processors, a training set comprising a plurality of images of subjects; identifying, by the one or more processors, a subset of the training set, wherein the subset comprises the image of the eye; identifying, by the one or more processors, in the image of the eye, a plurality of pixels corresponding to a lower eyelid portion of the eye; and generating, by the one or more processors, a statistical model for predicting a hemoglobin level from the plurality of pixels of the lower eyelid portion of the eye. In some cases, the identifying the subset of the training set operation is performed by a classification model. In further embodiments, the classification model is trained on a plurality of images, wherein a training image of the plurality of images comprises the image of the eye. In further embodiments, the eye comprises the lower eyelid portion and a sclera portion. In some cases, the classification model is trained to predict a presence of the eye in the image of the eye. In further embodiments, the eye in the image of the eye comprises a lower eyelid portion and a sclera portion. In further embodiments, the classification model is a fine-tuned computer vision model. In some cases, the identifying in the image of the eye operation is performed by a segmentation model. In further embodiments, the segmentation model comprises a semantic segmentation model. In further embodiments, the segmentation model is trained on a plurality of images, wherein an image of the plurality of images comprises the lower eyelid portion and a sclera portion. In some cases, the statistical model is trained using a plurality of images, wherein an image of the plurality of images comprises the lower eyelid portion. In some cases, the plurality of images comprises a generated image generated by a generative machine learning model. In further embodiments, the generated image comprises a generated lower eyelid portion. In some cases, the statistical model is a regression model. In some cases, the statistical model has a mean absolute error of at most about 0.93 grams per deciliter. In some cases, the statistical model has a confidence interval of at most about 1.56. In some cases, the statistical model uses a self-attention mechanism. In some cases, the method further comprises providing a data processing pipeline configured to perform operations comprising: filtering the image of the eye based on one or more of a yellow value, a red value, a brightness value, or a proportion contribution of the eye to the image of the eye; denoising the image of the eye; and adjusting a white balance of the image of the eye using a sclera portion of the eye. In some cases, the method further comprises generating, by the one or more processors, a statistical model for predicting a bilirubin level from the plurality of pixels of the lower eyelid portion of the eye. In some cases, the hemoglobin level is used to diagnose an anemia condition. In some cases, the method further comprises providing an anemia diagnosis using the hemoglobin level.

In yet another aspect disclosed herein is a computer-implemented method for analyzing an image of an eye, comprising: obtaining, by one or more processors, a training set comprising a first plurality of images, wherein an image of the first plurality of images comprises an eye of a subject; training, by the one or more processors, a machine learning model to predict a hemoglobin level of the subject from the eye of the subject using the first plurality of images; creating, by the one or more processors, a second plurality of images comprising the first plurality of images and one or more generated image, wherein the generated image is generated from a generative learning algorithm using the first plurality of images; and training, by the one or more processors, the machine learning model to predict the hemoglobin level using the second plurality of images. In some cases, the one or more generated image comprises a low hemoglobin level or a high hemoglobin level.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents and patent applications incorporated by reference contradict the disclosure contained in the specification, the specification is intended to supersede or take precedence over any such contradictory material.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings (also “Figure” and “FIG.” herein), of which:

FIG. 1 shows a non-limiting example of a system for determining hemoglobin levels;

FIG. 2 shows a non-limiting example of a computer-implemented method for determining hemoglobin levels;

FIG. 3 shows a non-limiting example of a computer-implemented method for generating a statistical model to determine hemoglobin levels;

FIG. 4A shows a non-limiting example of a classification component of a machine learning pipeline for determining hemoglobin levels;

FIG. 4B shows a non-limiting example of a segmentation component of a machine learning pipeline for determining hemoglobin levels;

FIG. 4C shows a non-limiting example of a regression component of a machine learning pipeline for determining hemoglobin levels;

FIG. 5 shows a non-limiting example of a computer-implemented method of training a machine learning model to predict hemoglobin levels;

FIG. 6 shows a non-limiting example of a machine learning architecture for determining hemoglobin levels;

FIG. 7 shows a non-limiting example of a user interaction with an application implementing hemoglobin determination;

FIG. 8 shows a non-limiting example of an image processing pipeline;

FIG. 9 shows a non-limiting example of a non-limiting example of a computer system that is programmed or otherwise configured to the methods disclosed herein;

FIG. 10 shows a non-limiting example of a web/mobile application provision system providing browser-based or native mobile user interfaces;

FIG. 11 shows a non-limiting example of a cloud-based web/mobile application provision system comprising an elastically load balanced, auto-scaling web server and application server resources as well synchronously replicated databases;

FIG. 12 shows a non-limiting example of an application architecture implementing the methods disclosed herein;

FIG. 13A shows a non-limiting example of a confusion matrix for anemia determination for adult men; and

FIG. 13B shows a non-limiting example of a confusion matrix for anemia determination for adult women.

### DETAILED DESCRIPTION

Disclosed herein are systems, methods, computer-readable media, and techniques for determining a value for a diagnostic indicator from non-invasive medical evaluation techniques using machine learning. The techniques disclosed herein may use multi-modal data of subjects to provide personalized, preventative healthcare based on the non-invasive medical evaluation techniques. Using the input modalities of a non-augmented (e.g., no specialized hardware modifications) mobile device, the platform disclosed herein may associate a recorded characteristic of a subject with a medical phenomenon. The input modalities such as a screen, camera, or microphone of the mobile device may be able to capture historical, visual, or auditory information about the subject in a manner amenable to a custom machine learning pipeline. This machine learning pipeline may take as input multi-modal data (e.g., text, image, audio) and provide diagnostic insights for the subject. The diagnostic insights may take the form of a value, as with the prediction of a hemoglobin value from an image of an eye. Insights such as this may be used to diagnose subjects with medical conditions such as anemia, jaundice, conjunctivitis, or other disorder. Additionally, the implementation of the systems, the methods, the computer-readable media, and the techniques disclosed herein in mobile device compatible software may provide time-based tracking of medical conditions for health monitoring.

Advantageously, the systems, the methods, the computer-readable media, and the techniques disclosed herein may provide a platform for a wholistic process that correlates multi-modal medical information with various health conditions to promote personalized preventive healthcare. This platform may be compatible with mobile devices for the acquisition and analysis of images of subjects to provide medical diagnostic information. The acquired images may provide prophylaxis to subjects by providing actionable insights into existing or nascent medical conditions. The platform may provide particular utility for the quantification of hemoglobin levels, which may provide an indication of an anemic condition of a subject. Accordingly, this platform may be used to address issues related to access of medical evaluation, monitory, diagnosis, intervention, and prevention for those with limited access to healthcare services. This platform may be compatible with mobile devices and may be used with images captured via non-invasive image acquisition techniques (e.g., simple image of an eye, audio recording of a cough). Additionally, the compatibility of the systems, the methods, the computer-readable media, and the techniques disclosed herein with mobile devices may provide health monitoring to subjects which may enhance compliance with health monitoring and treatment. Early indicators of a deteriorating or unexpected health condition (e.g., low hemoglobin levels, high bilirubin levels) may enable the prevention or management of chronic diseases such as chronic kidney disease, diabetes, or cancer. Further, the systems, the methods, the computer-readable media, and techniques disclosed herein may streamline the patient evaluation, diagnosis, and treatment processes by reducing the amount of digital communications included in these processes. Accordingly, streamlining these processes may improve communication network efficiency and reduce network traffic and network congestion for medical service provider networks.

The systems, the methods, the computer-readable media, and the techniques disclosed herein provide health monitoring and evaluation using computer vision and deep learning to deliver a wholistic process that may correlate multi-modal medical information with a variety of health conditions to promote personalized preventive healthcare. The multi-modal medical information may comprise one or more (1) image of body parts such as eye lid, fingernails, toenails, tongue, palm, skin, hair, (2) recording of breathing, coughing, or talking, (3) or medical history information in multi-modal format. Further, in some cases, the platform disclosed herein may provide (1) storage of medical information, (2) personalized preventive recommendations, or (3) health tracking over time.

In some cases, platform disclosed herein may be used to detect a level of hemoglobin in a subject. The hemoglobin level may be used to indicate anemia in the subject. In some cases, the platform disclosed herein may be used to detect a level of bilirubin in a subject. Multi-modal data processing using the platforms disclosed herein may be used to provide the determination of hemoglobin or bilirubin. In some cases, the determination may be achieved using machine learning to evaluate images of conjunctiva pallor inside the lower eyelid and the sclera portion of an eye along with known medical information. In some cases, the known medical information may comprise a subject's medical history or a subject's demographic data. In some cases, the subject's demographic data may comprise age, sex, or residency information. Residency information may be used to indicate possible exposure to environmental pollutants (e.g., heavy metals in an industrial area). A subject's medical history may comprise a patient history, medications, pre-existing conditions, images (e.g., MRI, X-ray, camera), audio recordings (e.g., doctor voice transcription, audio file of a cough), or allergies.

Disclosed herein are computer systems and computer-implemented methods to analyze an image of an eye. Analysis of the image may comprise obtaining the image of the eye; and providing a machine learning pipeline configured to perform operations comprising identifying a lower eyelid portion in the image of the eye and determining a hemoglobin level from an input comprising the lower eyelid portion. Additional systems and computer-implemented methods are disclosed to analyze an image of an eye. Analysis of the image may comprise obtaining, by one or more processors, a training set comprising a plurality of images of subjects; identifying, by the one or more processors, a subset of the training set, wherein the subset comprises the image of the eye; identifying, by the one or more processors, in the image of the eye, a plurality of pixels corresponding to a lower eyelid portion of the eye; and generating, by the one or more processors, a statistical model for predicting a hemoglobin level from the plurality of pixels of the lower eyelid portion of the eye. Yet additional systems and computer-implemented methods are disclosed to analyze an image of an eye. Analysis of the image may comprise obtaining, by one or more processors, a training set comprising a first plurality of images, wherein an image of the first plurality of images comprises an eye of a subject; training, by the one or more processors, a machine learning model to predict a hemoglobin level of the subject from the eye of the subject using the first plurality of images; creating, by the one or more processors, a second plurality of images comprising the first plurality of images and one or more generated image, wherein the generated image is generated from a generative learning algorithm using the first plurality of images; and training, by the one or more processors, the machine learning model to predict the hemoglobin level using the second plurality of images.

### Examples of Computing Systems for Analyzing an Image of an Eye

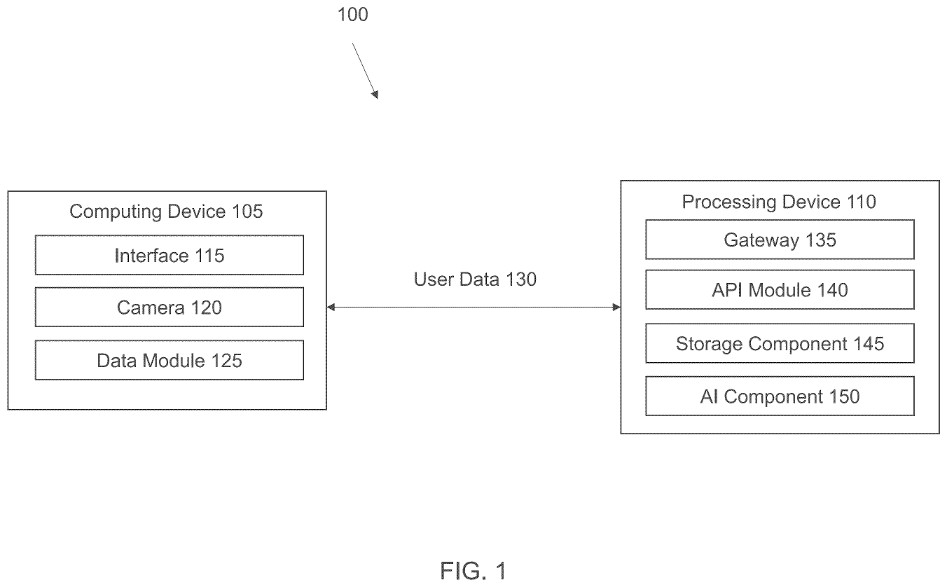

FIG. 1 depicts an example of a computing system 100 for performing analysis of an image of an eye. The computing system 100 may comprise a computing device 105 in communication with a processing device 110. The processing device may comprise an interface 115, a camera 120, and a data module 125. In some cases, the computing device 105 may transmit user data 130 to the processing device 110. The processing device 110 may be configured to receive the user data 130. In some cases, the processing device 110 may comprise a gateway 135. The processing device 110 may further comprise an API module 140, a storage component 145, or an artificial intelligence (AI) component 150.

In some cases, the computing device 105 may comprise a mobile device. The mobile device may comprise a smartphone, a smartwatch, a tablet computer, a personal computer, or an augmented/virtual reality device. In some cases, the computing device may comprise a camera 120 for obtaining an image from a user. For example, the camera 120 may be used to obtain an image of a body part such as an eye, a lower eyelid portion of an eye, a palm, a fingernail, a toenail, or a tongue. The image may be used as input into one or more machine learning model disclosed herein. The interface 115 of the computing device 105 may provide access to, for example, a portal, application, or website. The interface 115 may be used to provide insight into user history, user data, user notifications, or user recommendations. The interface 115 may further comprise an input component from which a user may upload or download data (e.g., images, medical records, account details). In some cases, the interface 115 may appear different for a patient and a healthcare provider. The computing device 105 may further comprise a data module 125 from which the user may interact with their data. For example, the user may select various presentations of their data. In some cases, the presentation may be a trend of patient data over time (e.g., hemoglobin over time-resolved measurements). The data module 125 may be used to show other summarizing or specific patient measurements or insights as disclosed herein. The data module 125 may be used to evaluate care recommendations for a user or a patient of a user. For example, data of a user may indicate that the user should implement one or more lifestyle or healthcare interventions to improve a condition (e.g., anemia).

The computing device 105 may be used to transmit user data 130 to the processing device 110. In some cases, the data may be encrypted before being transmitted to the processing device 110. In some cases, the data module 125 of the computing device 105 may comprise an encryption operation to ensure security of user data during transmission. Additionally, or alternatively, the user data 130 may be encrypted during transmission from the processing device 110 and de-encrypted on the computing device 105.

The processing device 110 may receive user data 130. In some cases, the processing device 110 may comprise one or more CPUs or GPUs. In some cases, the processing device 110 may comprise one or more cluster of CPUs or GPUs. In some cases, the processing device may comprise a gateway 135. The gateway 135 may be used to manage access and permissions. This may further ensure the protection of and security of user data 130 and other aggregated medical data stored on or accessible by the processing device 110. In some cases, the processing device 110 may comprise an API module for providing one or more application interfaces through which the computing device 105 and the processing device 110 may interact. API modules may comprise a data processing and storing module, a user creating module, a notification module, a recommendation generation module, a data cleaning module, a prediction result module, a trend module, or a scheduling module. In some cases, an API module may provide a means for organizing, processing, or presenting a finding, diagnosis, determination, or recommendation as disclosed herein. The processing device 110 may further comprise a storage component 145. The storage component 145 may provide both volatile (e.g., RAM) and non-volatile (e.g., ROM) storage media for storing medical data (e.g., user data 130). In some cases, the storage component 145 may comprise software instructions for obtaining data from a remote server. In some cases, the storage component 145 may comprise one or more local storage media or one or more remote storage media. In some cases, the data stored in the storage component 145 may be encrypted. The processing device 110 may further comprise an AI component 150. The AI component 150 may provide a machine learning pipeline for executing identification, classification, segmentation, regression, or recommendation operations. The AI component 150 may comprise one or more machine learning models as disclosed herein.

The computer system 100 may be used to execute one or more computer-implemented method for analyzing an image of an eye. The one or more computer-implemented method may additionally or alternatively be implemented on another computing system.

In some cases, a training operation may use one or more A100 GPU. In some cases, the training operation is performed on the processing device 110. In some cases, a CPU may be used for inference. The trained state of a model disclosed herein may be implemented by a CPU and used to generate hemoglobin estimation. The trained state of a model may be stored locally (e.g., on mobile device) or remotely (e.g., cloud). In some cases, the model (e.g., an identification, classification, regression, recommendation, segmentation model) may be stored on the computing device 105 or on the processing device 110.

### Examples of Inference Methods

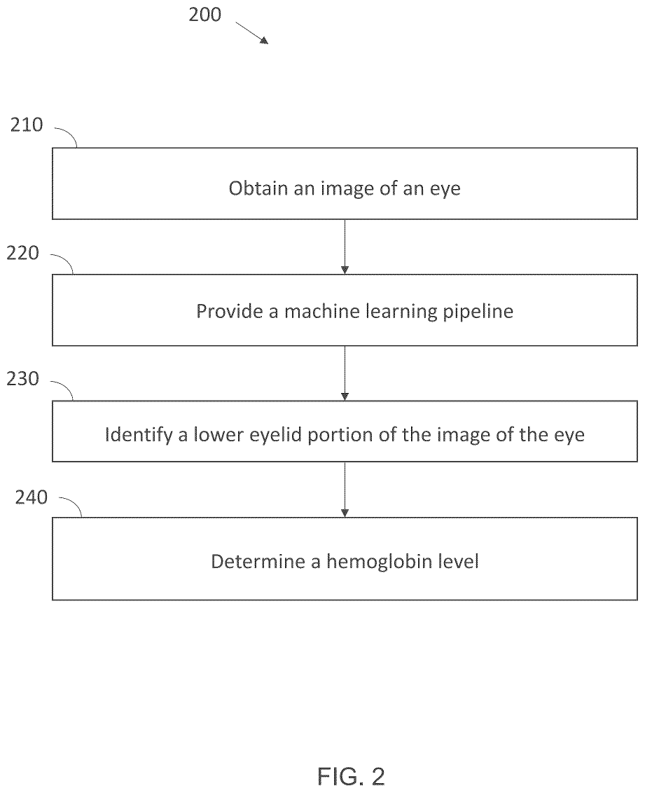

A computer-implemented method is shown in FIG. 2. The computer-implemented method 200 may comprise operations to analyze an image of an eye to determine a hemoglobin level. At operation 210, an image of an eye may be obtained. In some cases, an image of a left eye and an image of a right eye may be obtained. The image may be obtained using a mobile device. In some cases, the mobile device may be a non-augmented mobile device camera (e.g., Samsung Galaxy, Apple iPhone, Google Pixel, Apple iPad). The non-augmented smartphone may be used directly without additional hardware (e.g., attached lens, photometer).

In addition to obtaining the image, the mobile device may be used in evaluating, monitoring, or diagnosing a subject. A mobile device equipped with a camera and a processing device may be used to process and evaluate images for use in evaluating, monitoring, or diagnosing the subject. For instance, the mobile device may be used for validating input data, denoising input data, or hemoglobin estimation. Any one of or combination of these operations may be performed on the mobile device or on a cloud server (e.g., over the internet).

Operation 220 may provide a machine learning pipeline. The machine learning pipeline may be used to execute one or more machine learning operations. In some cases, the machine learning operations may be inferences into one or more trained machine learning models. The one or more machine learning operations may comprise one or more of a classification operation, a segmentation operation, or a regression operation. In some cases, operation 230 may comprise a classification operation and a segmentation operation to identify a lower eyelid portion of an image of an eye. In some cases, the classification operation may be used to identify a positive sample (e.g., an image containing a body part of interest). In some cases, the positive sample may comprise a lower eyelid portion. The segmentation operation may be used to identify a plurality of relevant pixels of the positive sample. For example, a portion of an image may comprise a subset of diagnostically relevant information of a larger image. Segmentation may be used to identify the pixels corresponding to this diagnostically relevant information. The regression operation may be used to determine value of interest (e.g., hemoglobin level). The machine learning operations may further comprise a classification or a recommendation. A classification operation may provide a distinct label for an input (e.g., a disease state such as “anemic”). A recommendation operation may be used to provide a care suggestion based on input data. For example, an image may be used to generate a suggestion that a user see a healthcare professional or adopt a lifestyle change to address an indicated or predicted disorder or disease indication.

In some cases, images may be used as a portion of an input for the evaluation of a subject. These images may, for example, be of a lower eyelid portion of an eye (e.g., the inner eyelid). The color of pallor of an eye may indicate the presence of a medical condition such as anemia, liver dysfunction, or conjunctivitis. In some cases, other body parts may be used as image input to evaluate the subject for medical conditions. Other body parts may include fingernails, toenails, tongue, palm, skin, or hair. Additionally, audio (e.g., cough or breathing audio) or text-based (e.g., questionnaire) based input may further inform the evaluation of the subject. Further, contextual data of the subject may further inform the presence of a medical condition of the subject. In some cases, the images may be acquired by a cell phone, a tablet, a laptop, a headset, or other mobile device. In some cases, the images may comprise an image of a left eye or an image of a left eye. Images of eyes may require exposure of the lower eyelid portion (e.g., inner eyelid). The images of the lower eyelid portion may be used to determine a hemoglobin level of the blood of the subject in operation 240. In some cases, the hemoglobin level may be determined in grams per deciliter.

In some cases, the determined hemoglobin value determined in operation 240 may be used to generate personalized care suggestions. For example, a comparison of the determined hemoglobin level versus a population statistic may indicate an anemic condition. Additionally, or alternatively, a comparison of the determined hemoglobin level versus a prior hemoglobin level of a subject may indicate an improvement or worsening of an anemia condition. The evaluation of a current anemia condition may be used to suggest lifestyle or medical interventions to manage an anemia condition.

In some cases, the contextual data of the subject may comprise anonymized demographic data. The anonymized demographic data may comprise, age, ethnicity, location, sex, or pregnancy status. Additionally, the contextual data of the subject may comprise a medical history. The medical history may comprise smoking, cancer, chemotherapy, chronic kidney disease, body temperature, fatigue, blood pressure, or diabetes. The medical history may comprise multi-modal data such as images (e.g., X-ray, MRI, PET Scan), medical recordings (e.g., audio), or text (e.g., medical report jacket).

In some cases, a machine learning model implemented in the platform disclosed herein to determine hemoglobin levels may obtain a mean absolute error (MAE) of at least about 0.5, 0.6, 0.7, 0.8, 0.9, 0.93, 1, 1.1, 1.2, 1.3, 1.4, 1.5, 1.6, 1.7, 1.8, 1.9, 2, or less. In some cases, the MAE of a hemoglobin model disclosed herein may be less than about 0.93 g/dL. In some cases, the MAE of a hemoglobin model disclosed herein may be less than about 1.1 g/dL. In some cases, the MAE of a hemoglobin model disclosed herein may be about 0.5 g/dL to about 2 g/dL. In some cases, the MAE of a hemoglobin model disclosed herein may be about 0.5 g/dL to about 1.1 g/dL. In some cases, the MAE of a hemoglobin model disclosed herein may be about 0.5 g/dL to about 0.6 g/dL, about 0.5 g/dL to about 0.7 g/dL, about 0.5 g/dL to about 0.8 g/dL, about 0.5 g/dL to about 0.9 g/dL, about 0.5 g/dL to about 1 g/dL, about 0.5 g/dL to about 1.1 g/dL, about 0.6 g/dL to about 0.7 g/dL, about 0.6 g/dL to about 0.8 g/dL, about 0.6 g/dL to about 0.9 g/dL, about 0.6 g/dL to about 1 g/dL, about 0.6 g/dL to about 1.1 g/dL, about 0.7 g/dL to about 0.8 g/dL, about 0.7 g/dL to about 0.9 g/dL, about 0.7 g/dL to about 1 g/dL, about 0.7 g/dL to about 1.1 g/dL, about 0.8 g/dL to about 0.9 g/dL, about 0.8 g/dL to about 1 g/dL, about 0.8 g/dL to about 1.1 g/dL, about 0.9 g/dL to about 1 g/dL, about 0.9 g/dL to about 1.1 g/dL, or about 1 g/dL to about 1.1 g/dL. In some cases, the confidence interval of the hemoglobin model disclosed herein may comprise a confidence interval of at least about 1, 1.1, 1.2, 1.3, 1.4, 1.5, 1.6, 1.7, 1.8, 1.9, 2, 2.1, 2.2, 2.3, 2.4, 2.5, or less. In some cases, the confidence interval of a hemoglobin model disclosed herein may be less than about 1.56 g/dL. In some cases, the confidence interval of the hemoglobin model disclosed herein may be about 1 g/dL to about 2 g/dL. In some cases, the confidence interval of the hemoglobin model disclosed herein may be about 1 g/dL to about 1.1 g/dL, about 1 g/dL to about 1.2 g/dL, about 1 g/dL to about 1.3 g/dL, about 1 g/dL to about 1.4 g/dL, about 1 g/dL to about 1.5 g/dL, about 1 g/dL to about 1.6 g/dL, about 1 g/dL to about 1.7 g/dL, about 1 g/dL to about 1.8 g/dL, about 1 g/dL to about 1.9 g/dL, about 1 g/dL to about 2 g/dL, about 1.1 g/dL to about 1.2 g/dL, about 1.1 g/dL to about 1.3 g/dL, about 1.1 g/dL to about 1.4 g/dL, about 1.1 g/dL to about 1.5 g/dL, about 1.1 g/dL to about 1.6 g/dL, about 1.1 g/dL to about 1.7 g/dL, about 1.1 g/dL to about 1.8 g/dL, about 1.1 g/dL to about 1.9 g/dL, about 1.1 g/dL to about 2 g/dL, about 1.2 g/dL to about 1.3 g/dL, about 1.2 g/dL to about 1.4 g/dL, about 1.2 g/dL to about 1.5 g/dL, about 1.2 g/dL to about 1.6 g/dL, about 1.2 g/dL to about 1.7 g/dL, about 1.2 g/dL to about 1.8 g/dL, about 1.2 g/dL to about 1.9 g/dL, about 1.2 g/dL to about 2 g/dL, about 1.3 g/dL to about 1.4 g/dL, about 1.3 g/dL to about 1.5 g/dL, about 1.3 g/dL to about 1.6 g/dL, about 1.3 g/dL to about 1.7 g/dL, about 1.3 g/dL to about 1.8 g/dL, about 1.3 g/dL to about 1.9 g/dL, about 1.3 g/dL to about 2 g/dL, about 1.4 g/dL to about 1.5 g/dL, about 1.4 g/dL to about 1.6 g/dL, about 1.4 g/dL to about 1.7 g/dL, about 1.4 g/dL to about 1.8 g/dL, about 1.4 g/dL to about 1.9 g/dL, about 1.4 g/dL to about 2 g/dL, about 1.5 g/dL to about 1.6 g/dL, about 1.5 g/dL to about 1.7 g/dL, about 1.5 g/dL to about 1.8 g/dL, about 1.5 g/dL to about 1.9 g/dL, about 1.5 g/dL to about 2 g/dL, about 1.6 g/dL to about 1.7 g/dL, about 1.6 g/dL to about 1.8 g/dL, about 1.6 g/dL to about 1.9 g/dL, about 1.6 g/dL to about 2 g/dL, about 1.7 g/dL to about 1.8 g/dL, about 1.7 g/dL to about 1.9 g/dL, about 1.7 g/dL to about 2 g/dL, about 1.8 g/dL to about 1.9 g/dL, about 1.8 g/dL to about 2 g/dL, or about 1.9 g/dL to about 2 g/dL. In some cases, the hemoglobin model may predict a hemoglobin level within 1.56 g/dL of a true value for at least about 95% of predictions. In some cases, the confidence interval indicates a 95% probability that a predicted hemoglobin value comprises a value within at least about 1, 1.1, 1.2, 1.3, 1.4, 1.5, 1.6, 1.7, 1.8, 1.9, 2, 2.1, 2.2, 2.3, 2.4, or 2.5 grams per deciliter (g/dL) of a true hemoglobin value.

A predicted hemoglobin level as disclosed herein may be used to diagnose an anemia condition in a subject. In some cases, the predicted hemoglobin level may be compared to a threshold value to diagnose the anemia condition. The threshold value may be demographic dependent. For example, a male subject may require a different threshold than a female subject to assign an anemia condition. The male subject may be diagnosed as presenting with anemia if their predicted hemoglobin level is less than or equal to 13 g/dL. The female subject may be diagnosed as presenting with anemia if their predicted hemoglobin level is less than or equal to 12 g/dL. The threshold value used in practice may be selected on further demographic or medical history details of the subject. For example, a comorbid illness, pregnancy, genetic factors, population statistics, or other relevant factors may be used to establish the threshold for the subject, a demographic, or a population.

### Examples of Training Methods

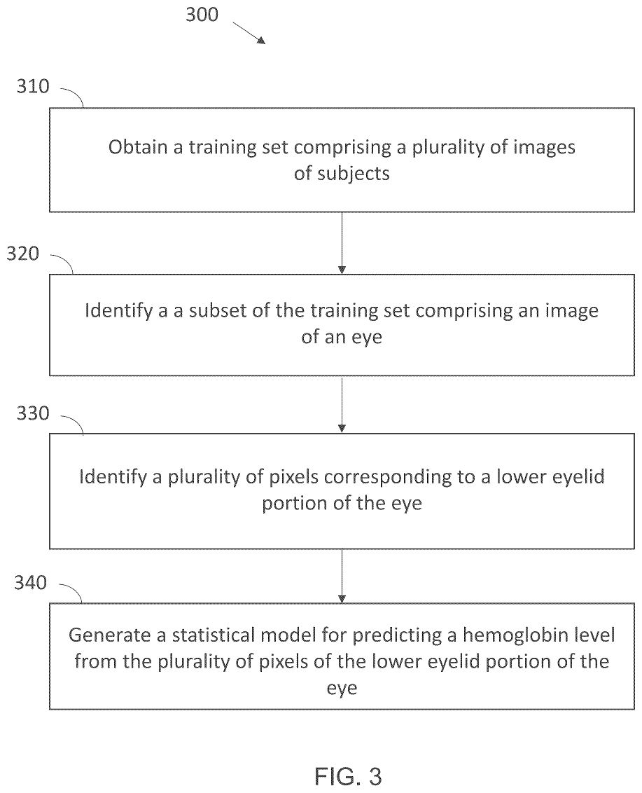

A computer-implemented method is shown in FIG. 3. The computer-implemented method 300 may comprise operations to analyze an image of an eye to determine a hemoglobin level. In some cases, the computer-implemented method 300 may be used to train one or more machine learning models. Operation 310 may comprise obtaining a training data set. The training dataset may comprise a plurality of images. A portion of the plurality of images may comprise an eye. For example, the training dataset may comprise positive samples that do contain an eye and negative samples that do not contain an eye. In some cases, the plurality of images may be cell phone images. A sample of the training dataset may comprise an image of the left eye or an image of the right eye. An image of the eye (e.g., the left eye, the right eye) may comprise a lower eyelid portion. In some cases, the lower eyelid portion is used for hemoglobin level determination. In some cases, the sample of the training dataset may further comprise demographic data (e.g., age, sex, location) or medical history data (e.g., diabetes, kidney disease, heart disease, etc.). In some cases, an image of the training dataset may be associated with a known hemoglobin level. In some cases, the known hemoglobin level may be obtained from a complete blood cell count measurement. The association of images of the training dataset with known hemoglobin levels may be used as features and labels for the training of a supervised learning algorithm (e.g., hemoglobin value regression).

Operation 320 may comprise identifying a subset of the training data comprising an image of an eye. In some cases, the operation 320 may comprising training a classification algorithm (e.g., a convolutional neural network, a residual neural network) to predict whether an image contains an eye. Additionally, or alternatively, the training may comprise training the classification algorithm to predict the presence of an eye comprising a lower eyelid portion and a sclera portion. In some cases, the classification algorithm may be trained from a non-trained or randomly initialized state. Training the classification algorithm may comprise using a custom dataset comprising labelled images. The labelled images may comprise positive samples (e.g., eye present) and negative sample (e.g., eye negative). In some cases, the classification algorithm may be trained to predict positive samples comprising both a lower eyelid portion and a sclera portion of an eye. In some cases, classification algorithm may be pre-trained to classify images into positive (e.g., eye containing) and negative (e.g., eye absent) images. In some cases, the training of the classification algorithm may comprise fine-tuning an existing image classification algorithm or leveraging an existing image encoder. A pre-trained image classification algorithm may comprise ResNet, VGG, Inception, MobileNet, or other model leveraging convolution. A pre-trained image classification may be trained on a large corpus of generic, labelled image data (e.g., ImageNet, CIFAR, MNIST). Fine-tuning a pre-trained algorithm (e.g., transfer learning) may comprise using labelled image data to teach a pre-trained computer vision algorithm to differentiate between positive and negative samples of the training dataset.

Operation 330 may comprise identifying relevant pixels from an image of the subset of the training set (e.g., a subset identified by operation 320). In some cases, the identification of relevant pixels may be performed by a segmentation algorithm. A segmentation algorithm may comprise predicting a class label for a pixel of an image (e.g., a lower eyelid portion pixel, a sclera portion pixel). The segmentation algorithm may be used to extract the pixels of an image that are relevant to a diagnosis or downstream prediction (e.g., hemoglobin level regression). In some cases, images may comprise a large portion of extraneous data that may hinder the learning of downstream algorithms. Segmentation algorithms may be used to refine the images of the subset of the training dataset. This may be done to reduce the amount of non-relevant visual data for hemoglobin level regression. In some cases, the segmentation algorithm is trained using the subset of the training set that comprises images of eyes. In some cases, the segmentation algorithm may be pre-trained and directly implemented in the pipeline. In some cases, the segmentation algorithm may be a semantic segmentation algorithm.

Operation 340 may comprise generating a statistical model for predicting a hemoglobin level. Operation 340 may use an input comprising a plurality of pixels identified as corresponding to a diagnostically relevant portion of an image. For example, the lower eyelid portion of an image (e.g., the pixels of the lower eyelid portion) may be used to determine a hemoglobin level of a subject whose eye is presented in the image. Operation 340 may take as input images or a plurality of pixels of lower eyelid portions of eyes and output hemoglobin levels. In some cases, the hemoglobin level may be provided in g/dL. In some cases, the hemoglobin level may be a label such as “anemic” or “not anemic.” In some cases, operation 340 may train a machine learning regression model to take as input images or a plurality of pixels of lower eyelid portions of eyes and output hemoglobin levels. The machine learning regression model may take as further input a medical history or demographic data of a user associated with the image to calculate hemoglobin levels.

In some cases, the platform disclosed herein may synthesize multi-modal data by processing and analyzing tens of thousands of connected data. The multi-modal data may be used to train one or more machine learning model. In some cases, the multi-modal data may comprise anonymized aggregate medical data. The multi-modal data may comprise images of eyes, lower eye portions, sclera of an eye. Images of eye or eye components (e.g., lower eyelid portion or sclera) may be used to hemoglobin level or bilirubin level. For instance, a conjunctiva pallor (pale lower eyelid portion) may indicate a presence of anemia in a subject. The hemoglobin level may be used to indicate a presence of an anemia condition. The bilirubin level may be used to indicate a liver disorder. The indications provided by the platform disclosed herein may be used for recommendation for anemia management, a suggestion to seek further evaluation, or a lifestyle change recommendation (e.g., diet change).

### Examples of Machine Learning Models

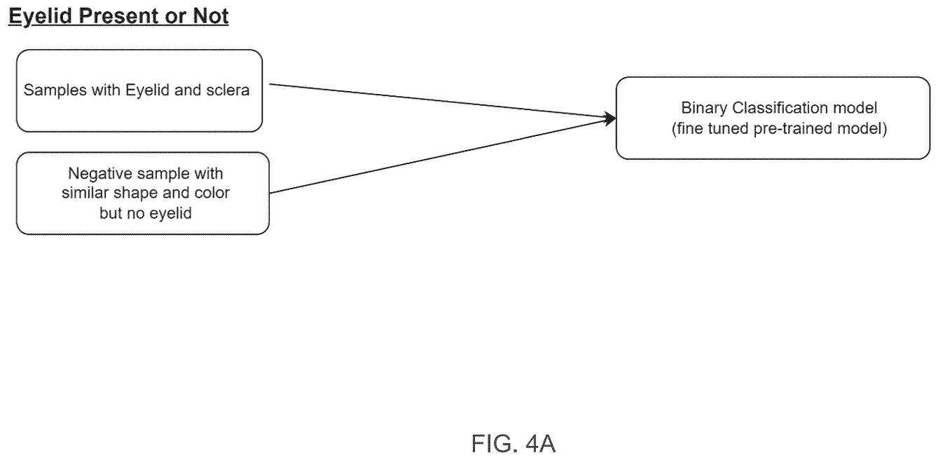

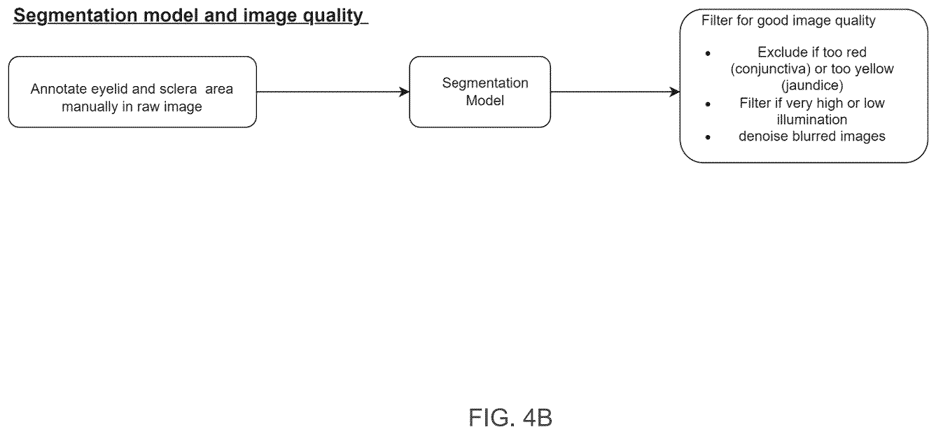

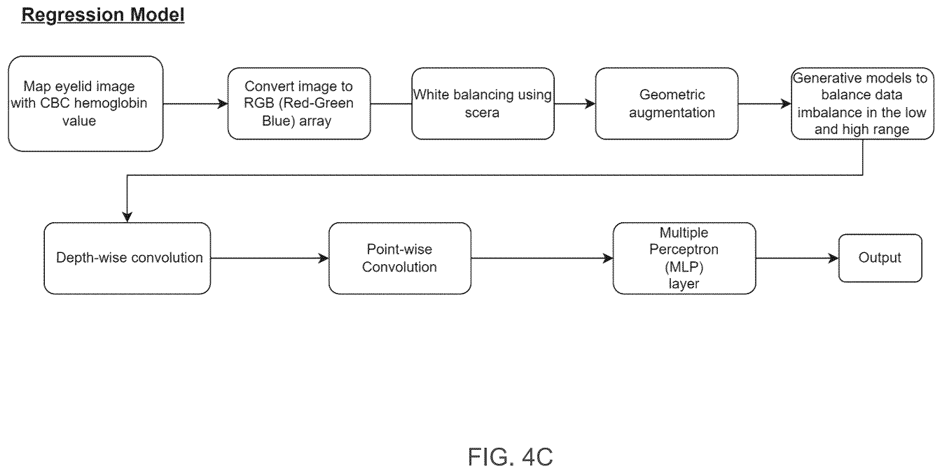

In some cases, as shown in FIG. 4A-4C, one or more machine learning models may be disposed in a pipeline of machine learning models. The machine learning pipeline may comprise three stages of image analysis. Additionally, or alternatively, a machine learning model of the machine learning pipeline may be used independently. (1) Stage one of the machine learning pipeline may comprise a classification algorithm as shown in FIG. 4A to determine a presence of a lower eyelid portion in an image. Training the classification algorithm may comprise a dataset containing labelled images. The images may comprise labels indicating the images as containing a lower eyelid portion and a sclera (positive sample) or not (negative sample). In some cases, a negative sample may comprise an object with a similar appearance to a positive sample. The classification algorithm may be a binary classification algorithm. In some cases, the classification algorithm may be a fine-tuned, pre-trained computer vision model. (2) Stage two of the machine learning pipeline may comprise an image segmentation algorithm as shown in FIG. 4B. Training the image segmentation algorithm may comprise a dataset comprising images with annotated pixels. In some cases, the annotation may be used to label portions of an eye of the image. For example, a pixel may be associated with one of a lower eyelid portion or a sclera portion. The segmentation model (e.g., a semantic segmentation model) may be used to identify a plurality of lower eyelid portion pixels of an image of an eye or a plurality of sclera portion pixels of an image of an eye. Additionally, the segmentation algorithm may be used to exclude values based on a red value, a yellow value, a brightness value, or a quality condition of the image. The segmentation algorithm may further be used to identify one or more relevant area in a partial image of an eye. In some cases, the identification of one or more relevant area in a partial image of an eye may leverage spatial relationships among the pixels of the image and local features of the image. Local features may comprise a color gradient or a portion of the eye (e.g., lower eyelid portion) or other statistical regularity that may not be perceivable or detectable by the human eye. In some cases, the statistical regularity may comprise a learnable feature of the image that is used by the segmentation model to perform the segmentation operation. (3) Stage three of the machine learning pipeline may comprise a regression algorithm as shown in FIG. 4C. Training the regression algorithm may comprise a dataset of a mapping between images of a lower eyelid portion of an image of an eye and a known CBC derived hemoglobin value. In some cases, the images of the lower eyelid portion of the image of the eye may be an output of the segmentation algorithm. The images of may be converted to a three-dimensional array of input data (e.g., height, width, color). Additionally, the white balance of the input image may be adjusted. The white balance may be adjusted versus the sclera portion of the input image. The input image may undergo a geometric augmentation. The geometric augmentation may comprise one or more transformation of the input image. The one or more transformation may comprise rotating, flipping, translating, or mirroring the input image. In some cases, the training dataset used to train the regression model may be supplemented with generated images. The generated images be an output of a generative machine learning model used to balance the training set. In some instances, machine learning models may be rendered more robust when trained on balanced training datasets. A balanced training set may comprise a uniform or near uniform representation of possible labels in the training set. During training (or inference), the input data may pass through one or more convolutional layers. The input data may be iteratively transformed during a forward pass through the convolutional layers such that the input data is transformed into a learned representation of the input data. The learned representation may be updated during a training operation or ultimately used in a final prediction in an inference operation. A forward pass through the convolutional layers may comprise a depth-wise convolution. The depth-wise convolution may be used to extract learned representation of the input data and may result in a change in dimensionality of the input data. The forward pass may further comprise of a point-wise convolution, which may be used to convert the at least three-dimensional learned representation of the input data into a shape compatible with input into a multiple layer perceptron (MLP) while retaining the information dense learned representation. The training operation may be used to associate the learned representation with an output. In some cases, the output may comprise a hemoglobin level (e.g., in g/dL). In some cases, the output may comprise a different value derived from the learned representation (e.g., bilirubin level, diagnosis class, anemia condition).

Image segmentation may be used to identify one or more pixel of an image. Image segmentation may comprise object segmentation, semantic segmentation, instance segmentation, or panoptic segmentation. The platform disclosed herein may comprise image segmentation to identify a plurality of pixels of a lower eyelid portion in an image of an eye of a subject. Additionally, or alternatively, the platform may identify a sclera portion of the image. In some cases, the platform may remove the sclera portion of the image to provide only the lower eyelid portion of the eye (e.g., for hemoglobin determination). In some cases, a red value of the pixels associated with the lower eyelid portion may be used to exclude images from a training set. For example, a red value satisfying a threshold may be used to indicate conjunctivitis of the eye, precluding an accurate hemoglobin determination. In some cases, a yellow value of the pixels associated with the lower eyelid portion may be used to exclude images from a training set. For example, a yellow value satisfying a threshold may be used to indicate a jaundice of the eye, precluding an accurate hemoglobin determination. In some cases, a white balance of a lower eyelid portion may be adjusted using the sclera portion of the eye. In some cases, the identification of the lower eyelid portion may be used to discard an image if the lower eyelid portion comprises to small a portion of the image. An image may be discarded based on relative lower eyelid portion representation if the lower eyelid portion comprises less than about 1%, 2%, 3%, 4%, 5%, 6%, 7%, 8%, 9%, 10%, 11%, 12%, 13%, 14%, 15%, 16%, 17%, 18%, 19%, 20% 25%, 30%, 35%, 40%, 50%, 55%, 60%, 70%, 80%, 90%, or 95% of the pixels of an image.

An image segmentation algorithm as disclosed herein may have an accuracy of at least about 80%, 85%, 90%, 91%, 92%, 93%, 94%, 95%, 96%, 97%, 98%, 99%, or 100%.

In some cases, machine learning (ML) may generally involve identifying and recognizing patterns in existing data in order to facilitate making predictions for subsequent data. ML may include a ML model (which may include, for example, a ML algorithm). Machine learning, whether analytical or statistical in nature, may provide deductive or abductive inference based on real or simulated data. The ML model may be a trained model. ML techniques may comprise one or more supervised, semi-supervised, self-supervised, or unsupervised ML techniques. For example, an ML model may be a trained model that is trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, large language models, vision transformers, or generative adversarial networks.

Training the ML model may include, in some cases, selecting one or more untrained data models to train using a training data set. The selected untrained data models may include any type of untrained ML models for supervised, semi-supervised, self-supervised, or unsupervised machine learning. The selected untrained data models may be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the ML model from the selected untrained data models may likewise be selected, such as limits on the ML model complexity or limits on the ML model refinement past a certain point. The ML model may be trained (e.g., via a computer system such as a server) using the training data set. In some cases, a first subset of the training data set may be selected to train the ML model. The selected untrained data models may then be trained on the first subset of training data set using appropriate ML techniques, based upon the type of ML model selected and any conditions specified for training the ML model. In some cases, due to the processing power requirements of training the ML model, the selected untrained data models may be trained using additional computing resources (e.g., cloud computing resources). Such training may continue, in some cases, until at least one aspect of the ML model is validated and meets selection criteria to be used as a predictive model.

In some cases, one or more aspects of the ML model may be validated using a second subset of the training data set (e.g., distinct from the first subset of the training data set) to determine accuracy and robustness of the ML model. Such validation may include applying the ML model to the second subset of the training data set to make predictions derived from the second subset of the training data. The ML model may then be evaluated to determine whether performance is sufficient based upon the derived predictions. The sufficiency criteria applied to the ML model may vary depending upon the size of the training data set available for training, the performance of previous iterations of trained models, or user-specified performance requirements. If the ML model does not achieve sufficient performance, additional training may be performed. Additional training may include refinement of the ML model or retraining on a different first subset of the training dataset, after which the new ML model may again be validated and assessed. When the ML model has achieved sufficient performance, in some cases, the ML may be stored for present or future use. The ML model may be stored as sets of parameter values or weights for analysis of further input (e.g., further relevant parameters to use as further predicted variables, further explanatory variables, further user interaction data, etc.), which may also include analysis logic or indications of model validity in some instances. In some cases, a plurality of ML models may be stored for generating predictions under different sets of input data conditions. In some cases, the ML model may be stored in a database (e.g., associated with a server).

### Examples of Analyzing Methods

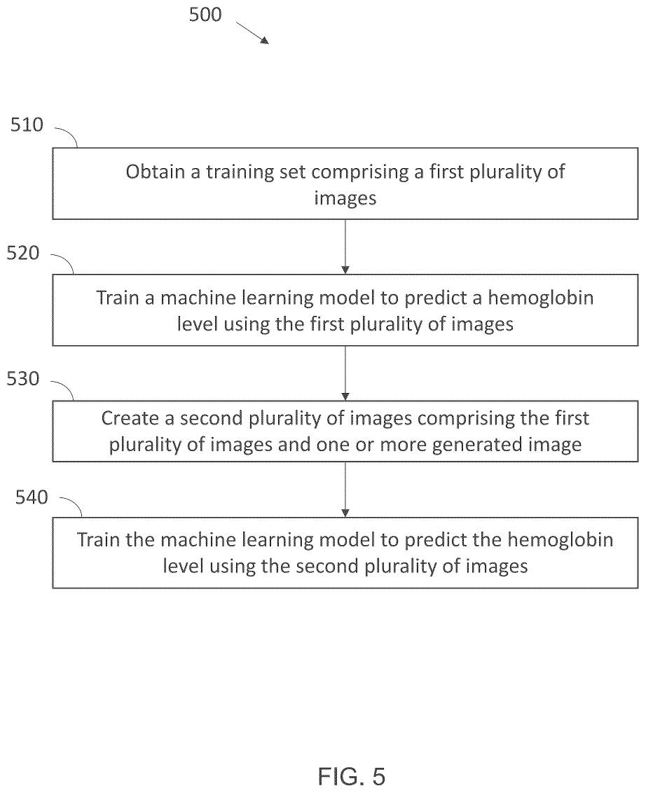

A computer-implemented method is shown in FIG. 5. The computer-implemented method 500 may comprise operations to analyze an image of an eye to determine a hemoglobin level. Operation 510 may comprise obtaining a training set comprising a first plurality of images. The plurality of images may comprise images of lower eye portions such as those identified in operation 330.

Operation 520 may comprise training a machine learning regression model to predict hemoglobin levels from an image of a lower eyelid portion. The machine learning regression model may learn to associate a pallor or color of a lower eyelid portion with a hemoglobin level (e.g., in g/dL). The machine learning regression model performance may depend on a quality of the training set. The quality of the training set may be dependent on the training set's representation of the possible types of input. In some cases, a training set may benefit from having a uniform or near uniform representation of input types and corresponding labels during training. For example, a data set with labels that form a normal distribution may not be suitable for training a machine learning model to predict values near the tail of the normal distribution (e.g., far from the mean). The state of the machine learning regression model after training during operation 520 may be improved by a second round of training. The composition of the training set (e.g., the first plurality of images) may be updated for a second round of training. This composition may be updated to increase the representation of training samples associated with difficult to predict labels (e.g., high values, low values). In some cases, the second round of training may be performed with a second plurality of images. The second plurality of images may comprise a portion of the first plurality of images. The second plurality of images may further comprise one or more images absent in the first plurality of images. The one or more images absent from the first plurality of images may comprise images of eyes with difficult to predict hemoglobin values (e.g., low or high hemoglobin).

In operation 530, a second plurality of images may be created to further refine the machine learning regression model. To improve the robustness of the machine learning regression model of operation of the computer-implemented method 500 or of other machine learning models of the platform disclosed herein, a dataset used for training one or more of the machine learning algorithms implemented herein may comprise one or more generated images. A generated image may comprise a new image or an augmentation of an existing image. In some cases, to balance the input data volume at lower and upper hemoglobin level, synthetic (generated) input data may be obtained using machine learning or non-machine learning techniques. For instance, non-machine learning methods may be used to obtain a larger dataset by rotating, scaling, shifting, translating, or color adjusting existing images of a dataset. Alternatively, or additionally, machine learning methods may be used to generate new training images from an existing dataset of training images. For instance, variational autoencoders (VAEs) or generative adversarial networks (GANs) may be used to produce entirely new training images from existing training images.

Operation 540 may comprise training the machine learning regression model using the second plurality of images comprising the generated images.

In some cases, a training set for training a classification model disclosed herein may comprise at least about 100, 1,000, 10,000, 100,000, 1,000,000, or more images. In some cases, a training set for training a segmentation model disclosed herein may comprise at least about 100, 1,000, 10,000, 100,000, 1,000,000, or more images. In some cases, a training set used for training a regression model disclosed herein may comprise at least about least 100, 1,000, 10,000, 100,000, 1,000,000, or more images.

### Examples of Data Processing

Images used as input into the platform disclosed herein may be pre-processed using denoising methods. Denoising methods may comprise using machine learning or non-machine learning methods. For example, encoder-decoder ML models, spatial domain filtering, or variational denoising methods may be used to improve input image quality. Types of de-noising operation used may comprise de-blurring, removing reflections, correcting color aberrations, or correcting brightness aberrations. Pre-processing during training may be used to exclude images. In some cases, images may be excluded based on a brightness value (e.g., too light or dark), a quality value (e.g., too blurry or noisy), a relevancy value (e.g., not containing a lower eyelid portion), or a color value which may indicate the presence of other conditions. For example, a high yellow value of a lower eyelid portion may indicate jaundice and therefore be excluded from a training dataset. Further, a high red value of a lower eyelid portion may indicate conjunctivitis and therefore be excluded from a training dataset.

In some cases, images may be post-processed. A hemoglobin value determined from a left eye may be expected to comprise a similar value as determined from a right eye. A large deviation between a left eye hemoglobin value and a right eye hemoglobin value may be used to prompt a subject to obtain a new image of each eye. In some cases, a deviation between a left eye and a right eye hemoglobin level may be used to prompt the subject when the deviation is greater than about 0.1, 0.2, 0.3, 0.4, 0.5, 0.6, 0.7, 0.8, 0.9, 1.0, 1.1, or more grams per deciliter.

### Examples of Model Architectures

The platform disclosed herein may be used to collect data of a plurality of subjects over time. This data may be used to further validate and refine the machine learning models employed in the platform. In some cases, collected data is verified for accuracy and used to fine-tune the models disclosed herein to improve their prediction accuracy.

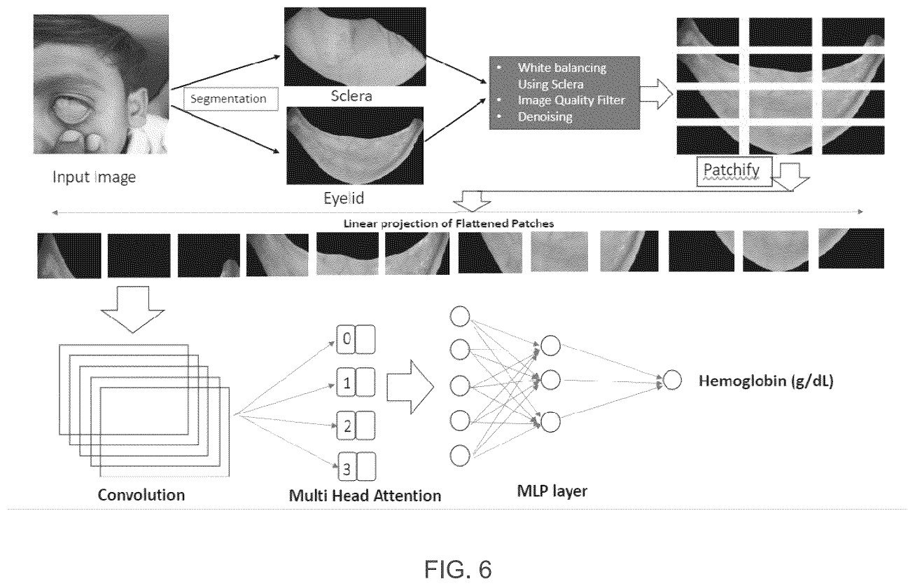

In some cases, as shown in FIG. 6, a machine learning pipeline may comprise a segmentation and a regression machine learning algorithm. The machine learning pipeline may further comprise one or more data processing operations. The machine learning pipeline may use an image comprising an eye as input. In some cases, the input may further comprise medical information or demographic information. The medical information or the demographic information may be multi-modal (e.g., text, images, audio). The segmentation operation may include identifying a plurality of pixels corresponding to one or more classes of object in the image. In some cases, the classes may be at least one or both of a lowery eyelid portion and a sclera portion. For some diagnoses, the lower eyelid portion may comprise the desired information (e.g., a determinable hemoglobin level). In such a case, the segmentation machine learning model may be used to identify and isolate a plurality of pixels corresponding to the lower eyelid portion. These pixels, which may constitute a portion of the image input, may be subjected to one or more post-processing operations including (1) a white balancing operation using the sclera portion of the eye, (2) a denoising operation, or (3) an image quality filtering operation (e.g., exclusion of photos based on color or brightness values). The lower eyelid portion data may be split into one or more sub-array of pixels for conversion into a series of sub-arrays. These sub-arrays may be exposed to one or more convolutional layers for the extraction of a learned representation of the input lower eyelid data. The learned representation may be passed into a multi-head attention layer. The multi-head attention layer may leverage self-attention to learn complex, spatially resolved associations in the learned representation. The multi-head attention layer may provide particular utility in considering the full scope of a visual or multi-modal input when determining a value (e.g., hemoglobin level). For instance, the plurality of pixels of the lower eyelid portion may comprise valuable holistic information that may be misconstrued by other computer vision algorithms that may assign meaning to the organization of image features and render the algorithms sensitive to perturbations in the learned features. In some cases, the multi-head attention layer may be used to retain one or more relationship among the pixels in an anterior and posterior region of the image. The retention of the relationship among the pixels may capture features associated with the color gradient of a lower eyelid portion of the eye. The formation of the sub-arrays of pixels of the image may change the relationship among portions of the lower eyelid portion of the image. The multi-head attention layer may leverage a self-attention mechanism to incorporate complex spatial relationships such as color gradient into a prediction.

The learned representation may be further transformed by a fully connected layer or layers and ultimately fed through an output layer to determine a final output value (e.g., hemoglobin level in g/dL).

The model for use in determining hemoglobin level may comprise a transformer layer. In some cases, the transformer layer comprises a self-attention mechanism. In some cases, the transformer layer comprises a multi-head attention mechanism. This model may comprise additional fully connected layers. This architecture may enable the use of the rich learned representation of multi-modal data for the prediction of a continuous value (e.g., hemoglobin value). In some cases, the architecture may be used for classification (e.g., classification of subject as anemic or not anemic).

### Examples of User Flow

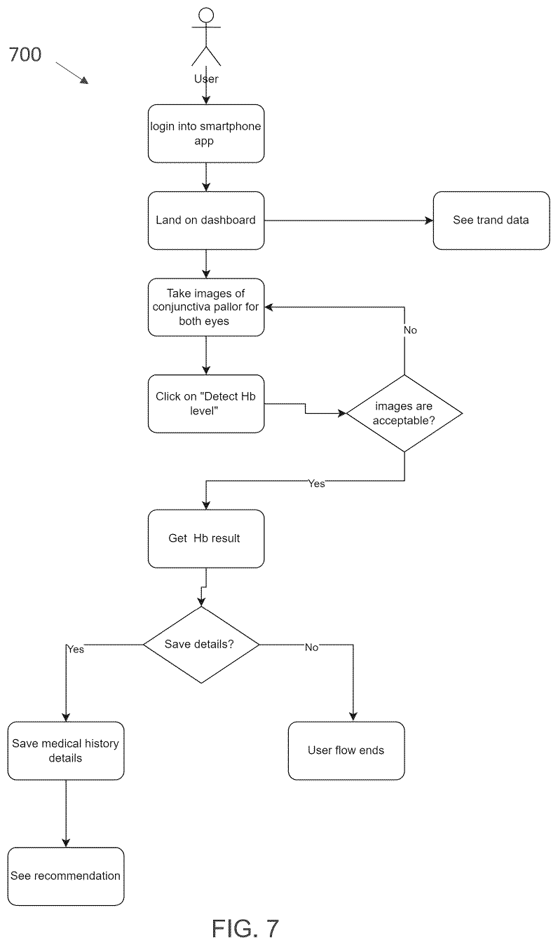

In some cases, as shown in FIG. 7, a user my interact with an application 700 implementing the techniques disclosed herein. In the application 700, a user (e.g., a patient, subject, healthcare provider) may log into a mobile device via a smartphone application. The user may see a dashboard comprising their personal data (e.g., demographic data, medical history, name, etc.). Their personal data may comprise trend information. The trend information may indicate changes to health status overtime (e.g., measured hemoglobin levels). This may provide particular utility in health monitoring and patient adherence to suggested health interventions. The application may prompt the user to take images of both eyes to measure the conjunctiva pallor their eyes. These images may be rejected, upon which time the user may be prompted to obtain replacement images, or the images may be accepted and used to determine a hemoglobin level of the user. Following the prediction of the hemoglobin level of the user, the user may choose to save or forego saving the obtained results. The user may also be presented with the results. The presentation of results may indicate a health condition of the subject (e.g., an anemia condition). The presentation may be accompanied with care suggestions. Care suggestions may comprise, for example, lifestyle changes or encouragement to seek further medical advice.

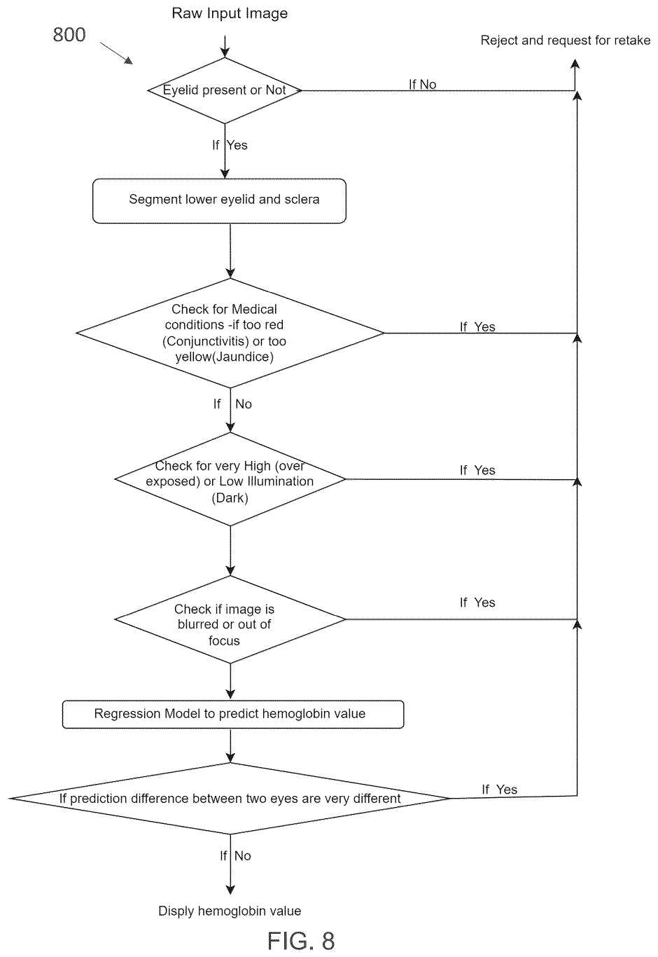

In some cases, as shown in FIG. 8, an image may be analyzed using a pipeline 800 as disclosed herein. In some cases, one or more image may be input into the platform disclosed herein. A machine learning model may be used to determine a presence of a lower eyelid portion of an eye of an input image. If the lower eyelid portion of the eye is determined to be absent, the platform may request a new picture to be obtained. The machine learning model may comprise a classification model (e.g., convolutional neural network, residual neural network). If they lower eyelid portion is predicted to be present, the image may be subjected to a segmentation algorithm. The segmentation algorithm may be used to identify a plurality of pixels of the image that contain the lower eyelid portion. In some cases, the segmentation algorithm may be a semantic segmentation algorithm trained to identify pixels of an image associated with a lower eyelid portion or a sclera portion of an eye. The lower eyelid portion may be evaluated for obfuscating conditions that may render the image not amenable to hemoglobin level determination. For example, the lower eyelid portion may be determined to comprise a red value or a yellow value that is indicative of conjunctivitis or jaundice. The presence of secondary conditions may make hemoglobin determination intractable. If the presence of a secondary condition is determined to be true, then the images may be rejected. The plurality of pixels may be further evaluated for a brightness value. If the brightness of an input image is determined to be too high (e.g., over-exposed) or too low (e.g., dark), then the image may be rejected and the platform may prompt the user to provide new photos. If the image input image is determined to comprise an acceptable brightness, then the image may be further checked for a blurry condition or a focus condition. A blurry or out of focus photo may result in a prompt to the user to provide a new image. Alternatively, the image be processed to improve the quality of the image (e.g., decrease blur). If the image is deemed to be sufficiently clear, the image (e.g., the plurality of pixels corresponding to the lower eyelid portion) may be used to determine a hemoglobin level of a user. In some instances, a hemoglobin level determined from a left eye may be compared with a hemoglobin value determined by a right eye. A large difference between predicted hemoglobin values may prompt the user to obtain one or more new images. If the difference in predicted hemoglobin values is determined to be below a threshold, then the result may be displayed to the user.

### Examples of Computer Implementations

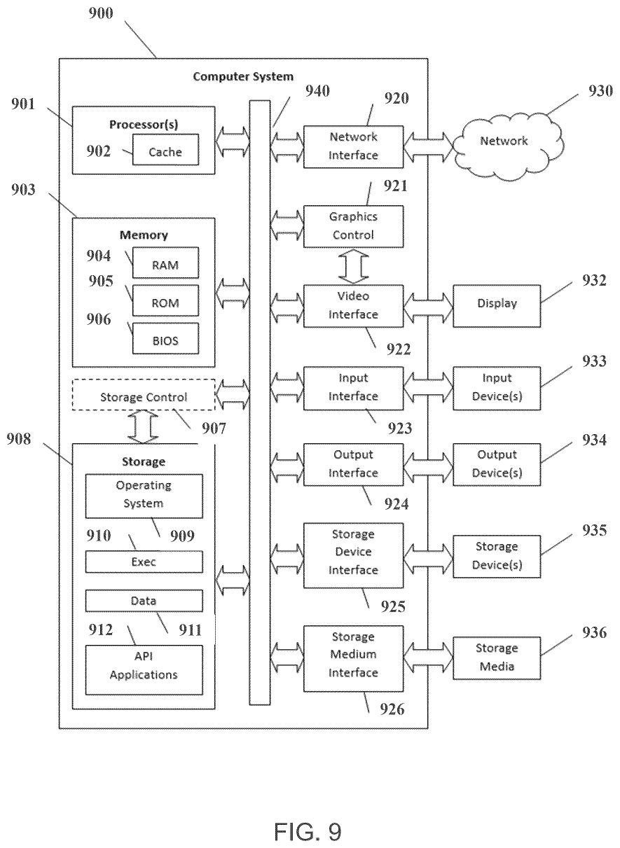

Referring to FIG. 9, a block diagram is shown depicting an exemplary machine that includes a computer system 900 (e.g., a processing or computing system) within which a set of instructions can execute for causing a device to perform or execute any one or more of the aspects or methodologies for static code scheduling of the present disclosure. The components in FIG. 9 are examples only and do not limit the scope of use or functionality of any hardware, software, embedded logic component, or a combination of two or more such components implementing particular functions. The computer system 900 may comprise or communicate with a computer system implementing the computer-implemented methods disclosed herein (e.g., the computing system 100 as shown in FIG. 1). In some cases, the computer system 900 may comprise the computing device 105 or components of the computing device 105 as a portion of the computer system 900. For example, the input interface 923 may comprise the interface 115. In a further example, the data module 125 may be implemented by the processor(s) 901 to interact with data 911 (e.g., data 911 comprising user data 130). Additionally, or alternatively, the processing device 110 of the computing system 100 may communicate with an implementation of the computer system 900. For example, the computing system 900 may interact with the processing device 110 by the network 930.

Computer system 900 may include one or more processors 901, a memory 903, and a storage 908 that communicate with each other, and with other components, via a bus 940. The bus 940 may also link a display 932, one or more input devices 933 (which may, for example, include a keypad, a keyboard, a mouse, a stylus, etc.), one or more output devices 934, one or more storage devices 935, and various tangible storage media 936. All of these elements may interface directly or via one or more interfaces or adaptors to the bus 940. For instance, the various tangible storage media 936 can interface with the bus 940 via storage medium interface 926. Computer system 900 may have any suitable physical form, including one or more integrated circuits (ICs), printed circuit boards (PCBs), mobile handheld devices (such as mobile telephones or PDAs), laptop or notebook computers, distributed computer systems, computing grids, or servers.

Computer system 900 includes one or more processor(s) 901 (e.g., central processing units (CPUs), general purpose graphics processing units (GPGPUs), or quantum processing units (QPUs)) that carry out functions. Processor(s) 901 optionally contains a cache memory unit 902 for temporary local storage of instructions, data, or computer addresses. Processor(s) 901 are configured to assist in execution of computer readable instructions. Computer system 900 may provide functionality for the components depicted in FIG. 9 as a result of the processor(s) 901 executing non-transitory, processor-executable instructions embodied in one or more tangible computer-readable storage media, such as memory 903, storage 908, storage devices 935, or storage medium 936. The computer-readable media may store software that implements particular functions, and processor(s) 901 may execute the software. Memory 903 may read the software from one or more other computer-readable media (such as mass storage device(s) 935, 936) or from one or more other sources through a suitable interface, such as network interface 920. The software may cause processor(s) 901 to carry out one or more processes or one or more steps of one or more processes described or illustrated herein. Carrying out such processes or steps may include defining data structures stored in memory 903 and modifying the data structures as directed by the software.

The memory 903 may include various components (e.g., machine readable media) including a random access memory component (e.g., RAM 904) (e.g., static RAM (SRAM), dynamic RAM (DRAM), ferroelectric random access memory (FRAM), phase-change random access memory (PRAM), etc.), a read-only memory component (e.g., ROM 905), and any combinations thereof. ROM 905 may act to communicate data and instructions unidirectionally to processor(s) 901, and RAM 904 may act to communicate data and instructions bidirectionally with processor(s) 901. ROM 905 and RAM 904 may include any suitable tangible computer-readable media described below. In one example, a basic input/output system 906 (BIOS), including basic routines that help to transfer information between elements within computer system 900, such as during start-up, may be stored in the memory 903.

Fixed storage 908 is connected bidirectionally to processor(s) 901, optionally through storage control unit 907. Fixed storage 908 provides additional data storage capacity and may also include any suitable tangible computer-readable media described herein. Storage 908 may be used to store operating system 909, executable(s) 910, data 911, applications 912 (application programs), and the like. Storage 908 can also include an optical disk drive, a solid-state memory device (e.g., flash-based systems), or a combination of any of the above. Information in storage 908 may, in appropriate cases, be incorporated as virtual memory in memory 903.

In one example, storage device(s) 935 may be removably interfaced with computer system 900 (e.g., via an external port connector (not shown)) via a storage device interface 925. Particularly, storage device(s) 935 and an associated machine-readable medium may provide non-volatile or volatile storage of machine-readable instructions, data structures, program modules, or other data for the computer system 900. In one example, software may reside, completely or partially, within a machine-readable medium on storage device(s) 935. In another example, software may reside, completely or partially, within processor(s) 901.

Bus 940 connects a wide variety of subsystems. Herein, reference to a bus may encompass one or more digital signal lines serving a common function, where appropriate. Bus 940 may be any of several types of bus structures including a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 900 may also include an input device 933. In one example, a user of computer system 900 may enter commands or other information into computer system 900 via input device(s) 933. Examples of an input device(s) 933 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some cases, the input device is a Kinect, Leap Motion, or the like. Input device(s) 933 may be interfaced to bus 940 via any of a variety of input interfaces 923 (e.g., input interface 923) including serial, parallel, game port, USB, FIREWIRE, THUNDERBOLT, or any combination of the above.

In some cases, when computer system 900 is connected to network 930, computer system 900 may communicate with other devices, specifically mobile devices and enterprise systems, distributed computing systems, cloud storage systems, cloud computing systems, and the like, connected to network 930. Communications to and from computer system 900 may be sent through network interface 920. For example, network interface 920 may receive incoming communications (such as requests or responses from other devices) in the form of one or more packets (such as Internet Protocol (IP) packets) from network 930, and computer system 900 may store the incoming communications in memory 903 for processing. Computer system 900 may similarly store outgoing communications (such as requests or responses to other devices) in the form of one or more packets in memory 903 and communicated to network 930 from network interface 920. Processor(s) 901 may access these communication packets stored in memory 903 for processing.

Examples of the network interface 920 include, but are not limited to, a network interface card, a modem, and any combination thereof. Examples of a network 930 or network segment 930 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 930, may employ a wired or a wireless mode of communication. In general, any network topology may be used.

Information and data can be displayed through a display 932. Examples of a display 932 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 932 can interface to the processor(s) 901, memory 903, and fixed storage 908, as well as other devices, such as input device(s) 933, via the bus 990. The display 932 is linked to the bus 940 via a video interface 922, and transport of data between the display 932 and the bus 940 can be controlled via the graphics control 921. In some cases, the display is a video projector. In some cases, the display is a head-mounted display (HMD) such as a VR headset. In further cases, suitable VR headsets include, by way of examples, HTC Vive, Oculus Rift, Samsung Gear VR, Microsoft HoloLens, Razer OSVR, FOVE VR, Zeiss VR One, Avegant Glyph, Freefly VR headset, and the like. In still further cases, the display is a combination of devices such as those disclosed herein.

In addition to a display 932, computer system 900 may include one or more other peripheral output devices 934 including an audio speaker, a printer, a storage device, and any combinations thereof. Such peripheral output devices may be connected to the bus 940 via an output interface 924. Examples of an output interface 924 include, but are not limited to, a serial port, a parallel connection, a USB port, a FIREWIRE port, a THUNDERBOLT port, and any combinations thereof.

In addition or as an alternative, computer system 900 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

In some cases, the various illustrative logical blocks, modules, circuits, and algorithm steps described in connection with the systems, the methods, the computer-readable media, and the techniques disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the systems, the methods, the computer-readable media, and the techniques disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the systems, the methods, the computer-readable media, and the techniques disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. In some cases, televisions, video players, and digital music players with optional computer network connectivity are suitable for use in the system described herein. Suitable tablet computers, in various cases, include those with booklet, slate, or convertible configurations.

In some cases, the computing device includes an operating system configured to perform executable instructions. The operating system is, for example, software, including programs and data, which manages the device's hardware and provides services for execution of applications. In some cases, suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some cases, suitable personal computer operating systems include, by way of examples, Microsoft® Windows®, Apple® Mac OS X®, UNIX®, and UNIX-like operating systems such as GNU/Linux®. In some cases, the operating system is provided by cloud computing. In some cases, suitable mobile smartphone operating systems include, by way of examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. In some cases, suitable media streaming device operating systems include, by way of examples, Apple TV®, Roku®, Boxee®, Google TV®, Google Chromecast®, Amazon Fire®, and Samsung® HomeSync®. In some cases, suitable video game console operating systems include, by way of examples, Sony® PS3®, Sony® PS4®, Sony® PS5®, Microsoft® Xbox 360®, Microsoft® Xbox One, Microsoft® Xbox Series X, Microsoft® Xbox Series S, Nintendo® Wii®, Nintendo® Wii U®, Nintendo® Switch™, and Ouya®.

Another aspect of the disclosure herein describes a non-transitory, computer-readable medium comprising executable instructions, wherein when a processor, when executing the executable instructions, performs a method as described herein.

### Web Application

In some cases, a computer program includes a web application. In some cases, the web application utilizes one or more software frameworks and one or more database systems. In some cases, a web application is created upon a software framework such as Microsoft® NET or Ruby on Rails (RoR). In some cases, a web application utilizes one or more database systems including, by way of examples, relational, non-relational, object oriented, associative, XML, and document oriented database systems. In further cases, suitable relational database systems include, by way of examples, Microsoft® SQL Server, mySQL™, and Oracle®. A web application, in various cases, is written in one or more versions of one or more languages. A web application may be written in one or more markup languages, presentation definition languages, client-side scripting languages, server-side coding languages, database query languages, or combinations thereof. In some cases, a web application is written to some extent in a markup language such as Hypertext Markup Language (HTML), Extensible Hypertext Markup Language (XHTML), or extensible Markup Language (XML). In some cases, a web application is written to some extent in a presentation definition language such as Cascading Style Sheets (CSS). In some cases, a web application is written to some extent in a client-side scripting language such as Asynchronous Javascript and XML (AJAX), Flash® ActionScript, JavaScript, or Silverlight® In some cases, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some cases, a web application is written to some extent in a database query language such as Structured Query Language (SQL). In some cases, a web application integrates enterprise server products such as IBM® Lotus Domino®. In some cases, a web application includes a media player element. In various further cases, a media player element utilizes one or more of many suitable multimedia technologies including, by way of examples, Adobe® Flash®, HTML 5, Apple® QuickTime®, Microsoft® Silverlight®, Java™, and Unity®.

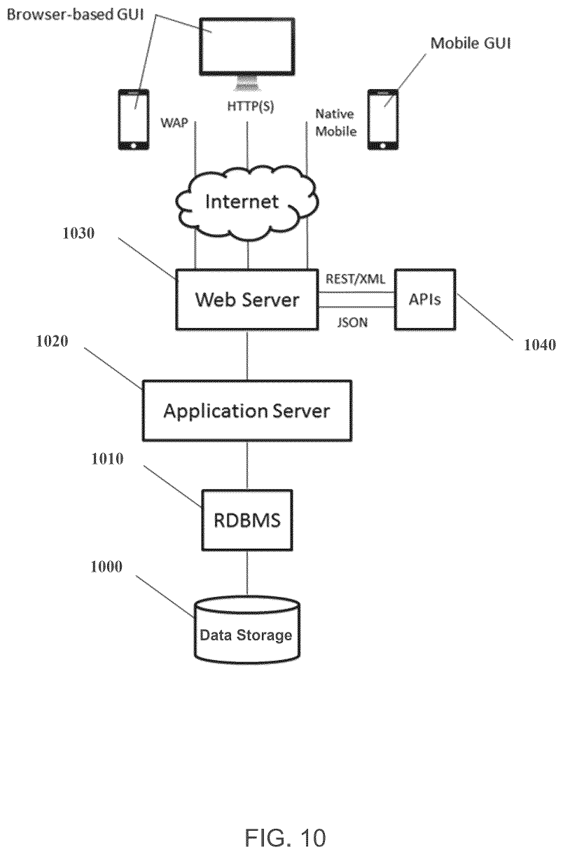

Referring to FIG. 10, in a particular example, an application provision system comprises one or more data storage system 1000 accessed by a relational database management system (RDBMS) 1010. Suitable RDBMSs include Firebird, MySQL, PostgreSQL, SQLite, Oracle Database, Microsoft SQL Server, IBM DB2, IBM Informix, SAP Sybase, Teradata, and the like. In this case, the application provision system further comprises one or more application severs 1020 (such as Java servers, .NET servers, PHP servers, and the like) and one or more web servers 1030 (such as Apache, IIS, GWS and the like). The web server(s) optionally expose one or more web services via app application programming interfaces (APIs) 1040. Via a network, such as the Internet, the system provides browser-based or mobile native user interfaces.

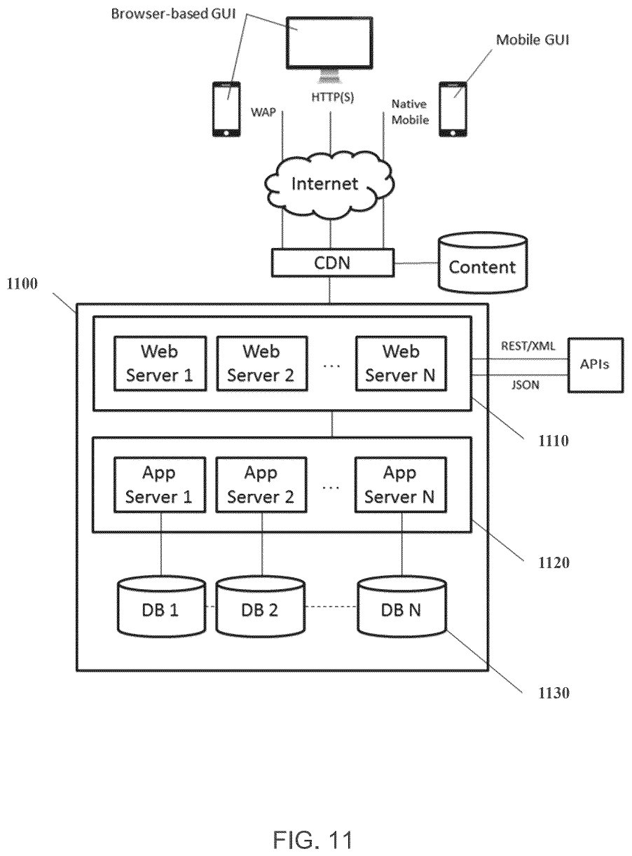

Referring to FIG. 11, in a particular example, an application provision system alternatively has a distributed, cloud-based architecture 1100 and comprises elastically load balanced, auto-scaling web server resources 1110 and application server resources 1120 as well synchronously replicated databases 1130.

### Mobile Application

In some cases, a computer program includes a mobile application provided to a mobile computing device. In some cases, the mobile application is provided to a mobile computing device at the time it is manufactured. In other cases, the mobile application is provided to a mobile computing device via the computer network described herein.

In view of the disclosure provided herein, a mobile application is created using a combination of hardware, languages, and development environments. In some cases, mobile applications may be written in several languages. Suitable programming languages include, by way of examples, C, C++, C #, Objective-C, Java™, JavaScript, Pascal, Object Pascal, Python™ Ruby, Rails, VB.NET, WML, and XHTML/HTML with or without CSS, or combinations thereof.

Suitable mobile application development environments are available from several sources. Commercially available development environments include, by way of examples, AirplaySDK, alcheMo, Appcelerator®, Celsius, Bedrock, Flash Lite, .NET Compact Framework, Rhomobile, and WorkLight Mobile Platform. Other development environments are available without cost including, by way of examples, Lazarus, MobiFlex, MoSync, and Phonegap. Also, mobile device manufacturers distribute software developer kits including, by way of examples, iPhone and iPad (iOS) SDK, Android™ SDK, BlackBerry® SDK, BREW SDK, Palm® OS SDK, Symbian SDK, webOS SDK, and Windows® Mobile SDK.

There may be several commercial forums are available for distribution of mobile applications including, by way of examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some cases, a computer program includes a standalone application, which is a program that is run as an independent computer process, not an add-on to an existing process, e.g., not a plug-in. In some cases, standalone applications may be compiled. A compiler is a computer program(s) that transforms source code written in a programming language into binary object code such as assembly language or machine code. Suitable compiled programming languages include, by way of examples, C, C++, Objective-C, COBOL, Delphi, Eiffel, Java™, Lisp, Python™, Visual Basic, and VB.NET, or combinations thereof. Compilation is often performed, at least in part, to create an executable program. In some cases, a computer program includes one or more executable complied applications.

### Web Browser Plug-In

In some cases, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Web browser plug-ins may include, Adobe® Flash® Player, Microsoft® Silverlight®, and Apple® QuickTime®. In some cases, the toolbar comprises one or more web browser extensions, add-ins, or add-ons. In some cases, the toolbar comprises one or more explorer bars, tool bands, or desk bands.

Plug-in frameworks may be available that enable development of plug-ins in various programming languages, including, by way of examples, C++, Delphi, Java™, PHP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of examples, Microsoft® Internet Explorer®, Mozilla® Firefox®, Google® Chrome, Apple® Safari®, Opera Software® Opera®, and KDE Konqueror. In some cases, the web browser is a mobile web browser. Mobile web browsers (also called microbrowsers, mini-browsers, and wireless browsers) are designed for use on mobile computing devices including, by way of examples, handheld computers, tablet computers, netbook computers, subnotebook computers, smartphones, music players, personal digital assistants (PDAs), and handheld video game systems. Suitable mobile web browsers include, by way of examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some cases, the systems, the methods, the computer-readable media, and the techniques disclosed herein include software, server, or database modules, or use of the same. Software modules may be generated embodying the concepts disclosed herein. The software modules disclosed herein are implemented in a multitude of ways. In various cases, a software module comprises a file, a section of code, a programming object, a programming structure, a distributed computing resource, a cloud computing resource, or combinations thereof. In further various cases, a software module comprises a plurality of files, a plurality of sections of code, a plurality of programming objects, a plurality of programming structures, a plurality of distributed computing resources, a plurality of cloud computing resources, or combinations thereof. In various cases, the one or more software modules comprise, by way of examples, a web application, a mobile application, a standalone application, and a distributed or cloud computing application. In some cases, software modules are in one computer program or application. In other cases, software modules are in more than one computer program or application. In some cases, software modules are hosted on one machine. In other cases, software modules are hosted on more than one machine. In further cases, software modules are hosted on a distributed computing platform such as a cloud computing platform. In some cases, software modules are hosted on one or more machines in one location. In other cases, software modules are hosted on one or more machines in more than one location.

### Databases

In some cases, the systems, the methods, the computer-readable media, and the techniques disclosed herein include one or more databases, or use of the same. In view of the disclosure provided herein many databases may be suitable for storage and retrieval of user data (e.g., user data 130), or any combination thereof. In various cases, suitable databases include, by way of examples, relational databases, non-relational databases, object oriented databases, object databases, entity-relationship model databases, associative databases, XML databases, document oriented databases, and graph databases. Further examples include SQL, PostgreSQL, MySQL, Oracle, DB2, Sybase, and MongoDB. In some cases, a database is Internet-based. In further cases, a database is web-based. In still further cases, a database is cloud computing-based. In a particular example, a database is a distributed database. In other cases, a database is based on one or more local computer storage devices.

### Data Transmission

The subject matter described herein, including methods and systems as described herein and may be configured to be performed in one or more facilities at one or more locations. Facility locations are not limited by country and include any country or territory. In some instances, one or more steps are performed in a different country than another step of the method. In some cases, one or more method steps involving a computer system are performed in a different country than another step of the methods provided herein. In some cases, data processing and storage are performed in a different country or location than one or more steps of the methods described herein. In some cases, one or more products or data are transferred from one or more of the facilities to one or more different facilities for analysis or further analysis. Data includes, but is not limited to, information regarding the stratification of a subject, and any data produced by the methods disclosed herein. In some cases of the methods and systems described herein, the subject information is compiled, and a subsequent data transmission step will transmit or store the subject information.

In some cases, any step of any method described herein is performed by a software program or module on a computer. In additional or further cases, data from any step of any method described herein is transferred to and from facilities located within the same or different countries, including analysis performed in one facility in a particular location and the data shipped to another location or directly to an individual in the same or a different country. In additional or further cases, data from any step of any method described herein is transferred to or received from a facility located within the same or different countries, including analysis of a data input, such as queries, objects, properties, types, filters, tables, or any combination thereof, performed in one facility in a particular location and corresponding data transmitted to another location.

### EXAMPLE APPLICATIONS

The following examples are included for illustrative purposes only and are not intended to limit the scope of the inventive concepts.

### Example 1: Application Implementation of Disclosed Platform

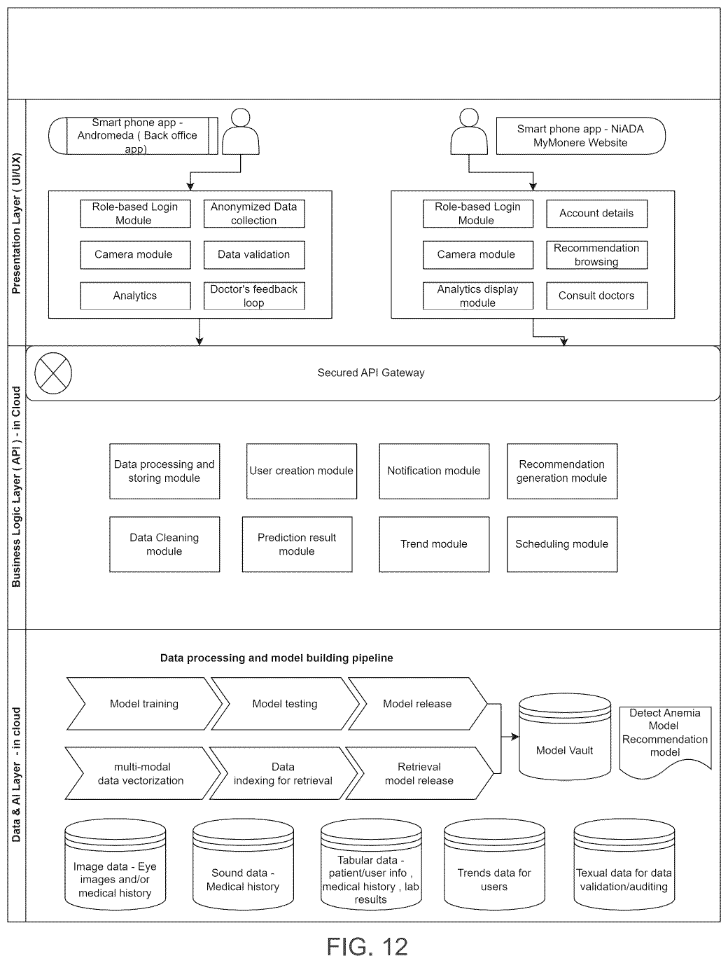

An example of a software implementation of the platform disclosed herein is shown in FIG. 12. A presentation layer is provided to a user via a mobile device application or a web application. Included in the presentation layer are role-based login modules to determine the appropriate interface to display to the user. A healthcare provider may, for instance, have access to aggregate anonymized data, a data validation module, and doctor's feedback loop module. Alternatively, a user may have an account details, care suggestion recommendation browsing, and doctor consultation features presented to them. Regardless of user, a camera module is presented for data collection.

Data access by the healthcare professional or the user will pass through a secured API gateway to ensure patient data privacy and security. Following passage through this gateway, the user or data can interact with a variety of data processing modules via a business logic layer (e.g., API). In this layer, there is (1) a data processing and storage module to manage data intake and retention, (2) a user creation module that can be used to open or manage user accounts, (3) a notification module that can be used to issue alerts, messages, or notices to users, (4) a recommendation generation module that can be used to issue advice or suggestions to users on how to manage any stored or determined health conditions, (5) a data cleaning module to ensure data quality and appropriateness, (6) a prediction result module to generate and present predictions from user data, (7) a trend module to track and present health data evolution over time, and (8) a scheduling module to help monitor and enforce user compliance with health interventions or recommendations.

Under the API layer is a data and AI layer, which is additionally embodied on one or more cloud servers. The data and API layer is used for model training, model testing, model release, multi-modal data vectorization, data indexing for retrieval, and model retrieval/release. Models used or developed by the data and AI layer are stored in a model vault. The vault may store the various models relied on by the platform as well as various model versions for model backups. One model contained in the model vault is the anemia detection model and the recommendation model. These models may be used to determine hemoglobin levels in blood and provide care suggestions based on determined hemoglobin levels. In addition to the model vault, a number of databases exist for various data needs of the platform. In this example, medical image data, sound data, tabular data/user info (e.g., lab results), trend data for users (e.g., aggregate anonymized data), and textual data for data validation and auditing are each stored in a database.

### Example 2: Demographic Dependent Classification Algorithm Confusion Matrices

An algorithm for predicting an anemia condition in a subject was trained as disclosed herein. A positive anemia condition was assigned to men based on a predicted hemoglobin level of less than or equal to 13 g/dL. A positive anemia condition was assigned to women based on a predicted hemoglobin level of less than or equal to 12 g/dL. The classification metrics as described below were calculated. The values for the classification metrics below were dependent on the selected threshold values and may change depending on demographics or learned ideal threshold values for accurately predicting an anemia condition.

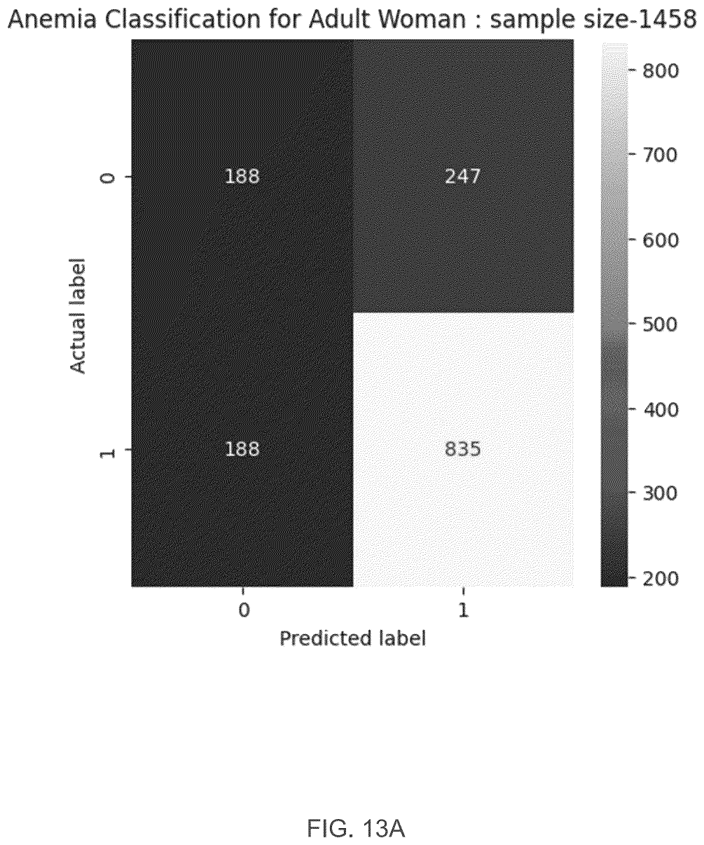

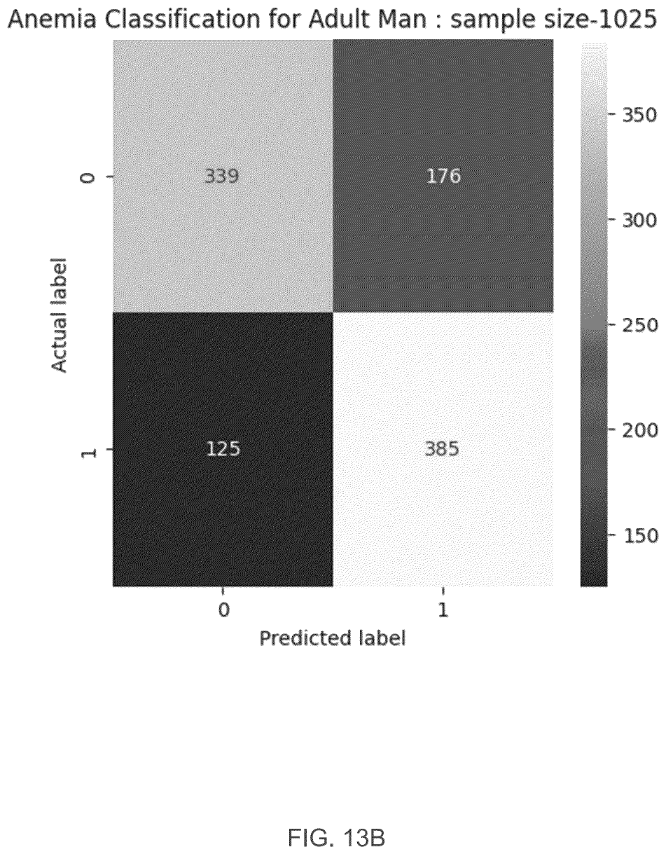

A confusion matrix is shown in FIG. 13A for the prediction of an anemia condition in a sample of 1,458 adult women. The precision for positive anemia condition was determined to be 0.772. The recall for positive anemia condition was determined to be 0.816. The precision for normal (negative anemia condition) was determined to be 0.5. The recall for normal was determined to be 0.432. The overall accuracy for classifying anemia condition was determined to be 0.702.

A confusion matrix is shown in FIG. 13B for the prediction of an anemia condition in a sample of 1,025 adult men. The precision for positive anemia condition was determined to be 0.686. The recall for positive anemia condition was determined to be 0.755, The precision for normal (negative anemia condition) was determined to be 0.731. The recall for normal was determined to be 0.658. The overall accuracy for classifying anemia condition was determined to be 0.706.

### Certain Definitions and Additional Considerations

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity or for ready reference, and the inclusion of such definitions herein should not necessarily be construed to represent a substantial difference over what is generally understood in the art.

As used in this specification and the appended claims, the terms “machine learning,” “machine learning techniques”, “machine learning algorithm,” “machine learning operation,” and “machine learning model” generally refer to any system or analytical or statistical procedure that may progressively improve computer performance of a task. An example of such a task is to model the probabilistic relationship mathematically or computationally between an image of a lower eyelid portion and a hemoglobin level.

As used in this specification and the appended claims, “some embodiments,” “further embodiments,” or “a particular embodiment,” means that a particular feature, structure, or characteristic described in connection with the embodiment is included in at least one embodiment. Thus, the appearances of the phrase “In some cases,” or “in further embodiments,” or “in a particular embodiment” in various places throughout this specification are not necessarily all referring to the same embodiment. Furthermore, the particular features, structures, or characteristics may be combined in any suitable manner in one or more embodiments.

As used in this specification and the appended claims, when the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

As used in this specification and the appended claims, when the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used in this specification, “or” is intended to mean an “inclusive or” or what is also known as a “logical OR,” wherein when used as a logic statement, the expression “A or B” is true if either A or B is true, or if both A and B are true, and when used as a list of elements, the expression “A, B or C” is intended to include all combinations of the elements recited in the expression, for example, any of the elements selected from the group consisting of A, B, C, (A, B), (A, C), (B, C), and (A, B, C); and so on if additional elements are listed. As such, any reference to “or” herein is intended to encompass “and/or” unless otherwise stated.

As used in this specification and the appended claims, the indefinite articles “a” or “an,” and the corresponding associated definite articles “the” or “the,” are each intended to mean one or more unless otherwise stated, implied, or physically impossible. Yet further, it should be understood that the expressions “at least one of A and B, etc.,” “at least one of A or B, etc.,” “selected from A and B, etc.” and “selected from A or B, etc.” are each intended to mean either any recited element individually or any combination of two or more elements, for example, any of the elements from the group consisting of “A,” “B,” and “A AND B together,” etc.

As used in this specification and the appended claims “about” or “approximately” may mean within an acceptable error range for the value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where values are described in the application and claims, unless otherwise stated the term “about” meaning within an acceptable error range for the particular value may be assumed.

While preferred embodiments of the present invention have been shown and disclosed herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention disclosed herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

It should be noted that various illustrative or suggested ranges set forth herein are specific to their example embodiments and are not intended to limit the scope or range of disclosed technologies, but, again, merely provide example ranges for frequency, amplitudes, FIG. associated with their respective embodiments or use cases. Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

It should be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based at least in part on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are disclosed herein as including logic or a number of routines, subroutines, applications, or instructions. These may constitute either software (e.g., code embodied on a machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as disclosed herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, hardware modules may encompass a tangible entity, be that an entity that is physically constructed, permanently configured (e.g., hardwired), or temporarily configured (e.g., programmed) to operate in a certain manner or to perform certain operations disclosed herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and may operate on a resource (e.g., a collection of information). Elements that are described as being coupled and or connected may refer to two or more elements that may be (e.g., direct physical contact) or may not be (e.g., electrically connected, communicatively coupled, etc.) in direct contact with each other, but yet still cooperate or interact with each other.

The various operations of example methods disclosed herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines disclosed herein may be at least partially processor-implemented. For example, at least some of the operations of a method may be performed by one or more processors or processor-implemented hardware modules. The performance of certain operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

It will be understood that, although the terms first, second, FIG. may be used herein to describe various elements, these elements should not be limited by these terms. These terms are only used to distinguish one element from another. For example, a first element may be termed a second element, and, similarly, a second element may be termed a first element, without departing from the scope of the present disclosure.

What is claimed is:

1-49. (canceled)

50. A method for training a machine learning model to analyze an image of an eye of a subject, comprising:
a. obtaining, by one or more processors, a first training set comprising a first plurality of images, wherein said first plurality of images comprises said image of said eye of said subject;
b. training, by said one or more processors, a machine learning model to predict a hemoglobin level of said subject from said eye of said subject using said first training set;
c. creating, by said one or more processors, a second training set comprising a second plurality of images, wherein said second plurality of images comprises one or more generated images, wherein said one or more generated images are generated by a generative learning algorithm trained on a plurality of images each comprising an eye; and
d. training, by said one or more processors, said machine learning model to predict said hemoglobin level using said second training set.

51. The method of claim 50, wherein said one or more generated images correspond to a low hemoglobin level or a high hemoglobin level.

52. The method of claim 50, wherein said image of said eye of said subject comprises image data of a lower eyelid portion and a sclera portion.

53. The method of claim 50, further processing said image of said eye of said subject, wherein processing said image of said eye of said subject comprises:
(i) filtering said image of said eye of said subject based on one or more of a yellow value, a red value, a brightness value, or a proportion contribution of said eye of said subject to said image of said eye of said subject;
(ii) denoising said image of said eye of said subject; or
(iii) adjusting a white balance of said image of said eye of said subject using a sclera portion of said eye of said subject.

54. The method of claim 50, further comprising generating said first plurality of images by:
(i) determining that each of said first plurality of images contains an eye using a classification model and removing any image that does not contain an eye, and
(ii) segmenting each of said first plurality of images using a segmentation model to identify a lower eyelid portion and a sclera portion.

55. The method of claim 50, further comprising training a classification model to predict the presence of an eye in each image of said first plurality of images.

56. The method of claim 50, further comprising training a segmentation model to identify a plurality of pixels corresponding to a lower eyelid portion of each said first plurality of images.

57. The method of claim 50, wherein each image of said first training set is associated with a known hemoglobin level.

58. The method of claim 50, wherein said one or more generated image comprises a generated lower eyelid portion.

59. The method of claim 50, wherein said second plurality of images comprises a near uniform distribution of possible labels in said first training set.

60. The method of claim 50, wherein said one or more generated image comprises a new image outside said first training set.

61. The method of claim 60, wherein said generative learning algorithm comprises one or both of a variational autoencoder (VAE) or a generative adversarial network (GAN).

62. The method of claim 50, further comprising performing operations (a)-(d) until said machine learning model is trained to predict hemoglobin levels to within about 1.56 grams per deciliter of a true hemoglobin level.

63. The method of claim 50, wherein said second plurality of images comprises an augmented image, wherein said augmented image is based on an image of said first plurality of images.

64. The method of claim 63, further comprising generating said augmented image by rotating, scaling, shifting, translating, or color adjusting said image of said first plurality of images.

65. The method of claim 50, wherein one or both of said first training set or second training set comprise medical information of said subject.

66. The method of claim 65, wherein said known medical information comprises one or both of demographic data or medical history.

67. The method of claim 50, further comprising providing said predicted hemoglobin level to an interface of a user device.

68. The method of claim 50, further comprising determining a hemoglobin level for a second subject based on an image of an eye of said second subject.

69. The method of claim 68, further comprising providing an indication of an anemic condition to said second subject and recommending a dietary change to said second subject to treat said anemic condition.
